(19) United States
(12) Patent Application Publication
(10) Pub. No.: US 2025/0179506 A1
(43) Pub. Date: Jun. 5, 2025

(54) MICROORGANISM STRAIN FOR ANTIBIOTIC-FREE PLASMID-BASED FERMENTATION AND METHOD FOR GENERATION THEREOF

(71) Applicant: Gen-H Genetic Engineering Heidelberg GmbH, Heidelberg (DE)

(72) Inventors: Harald KRANZ, Leimen (DE); Marlen SCHMIDT, Heidelberg (DE); Katherine Emily BRECHUN, Heidelberg (DE); Marion FOERSCHLE, Heidelberg (DE)

(21) Appl. No.: 18/842,950

(22) PCT Filed: Mar. 2, 2023
(86) PCT No.: PCT/EP2023/055347

§ 371 Date: Aug. 30, 2024

(30) Foreign Application Priority Data
Mar. 4, 2022 (EP) 22160310.3
Nov. 16, 2022 (EP) 22207908.9

(51) Int. Cl. C12N 15/63; C12N 15/70; C12P 1/04

(52) U.S. Cl. CPC C12N 15/635; C12N 15/70; C12P 1/04

(57) ABSTRACT

The present invention relates to means for recombinant manufacture. In particular, it relates to a recombinant bacterial cell comprising in its genome at least one essential gene the endogenous promoter of which has been replaced by a heterologous expression control sequence being inducible by an inducer molecule, such that the expression of said essential gene in the recombinant bacterial cell is dependent on the presence of said inducer molecule. Further contemplated are methods for generating the recombinant bacterial cell of the invention as well as a method for recombinant manufacture of a compound of interest. The present invention also provides for use the recombinant bacterial cell of the invention for recombinant manufacture of a compound of interest and a kit for manufacture of a compound of interest.

The present invention relates to means for recombinant manufacture. In particular, it relates to a recombinant bacterial cell comprising in its genome at least one essential gene the endogenous promoter of which has been replaced by a heterologous expression control sequence being inducible by an inducer molecule, such that the expression of said essential gene in the recombinant bacterial cell is dependent on the presence of said inducer molecule. Further contemplated are methods for generating the recombinant bacterial cell of the invention as well as a method for recombinant manufacture of a compound of interest. The present invention also provides for use the recombinant bacterial cell of the invention for recombinant manufacture of a compound of interest and a kit for manufacture of a compound of interest.

Plasmid-based expression of genes is an important tool for industrial-scale manufacture of chemical or pharmaceutical products. Typically, bacteria, such as E. coli, are transformed with an expression plasmid encoding a gene of interest required for the recombinant manufacture of a desired chemical or pharmaceutical product. Conventional cultivation methods require selection markers, such as resistance genes against antibiotics, in the expression plasmid and the use of antibiotics in the culture in order to select transformed bacteria and subsequently keep the expression plasmids stable in said transformed bacteria. However, the excessive use of antibiotics is expensive and causes various environmental and health problems, e.g., it facilitates the generation of multi-resistant microorganisms such as multi-resistant pathogens.

Several techniques have been developed for the recombinant manufacture of desired products which are independent of the use of antibiotics. For example, a toxin gene was introduced into a bacterial chromosome and will be expressed. Detoxification is achieved if a plasmid is introduced which in addition to a gene of interest also expresses a detoxification gene (Szpirer 2005).

In another approach, a conditional essential gene dapD is downregulated by a repressor. Only if an expression plasmid is introduced that comprises a gene of interest and allows for removal of the repressor due to the presence of an operator, which competes in binding the repressor (so named repressor titration technique) and the bacterial can be propagated (Cranenburgh 2001). This approach needs as a limitation the existence of the operator on a high copy number plasmids to titrate the repressor in such a manner that the bacterium can survive and for the necessary dapD gene expression. In addition, dapD is under transcriptional control of the lac operator/promoter (lacO/P) which dramatically limits the use of this system. The widely used T7 expression system is not compatible with this system. Furthermore, Cranenbourg's approach requires a complex, multi-stage process to create the expression strains.

RNA-based selection markers have been developed. In such an approach, a marker on a chromosome will be neutralized by an antisense RNA expressed from the successfully transformed expression plasmid comprising the gene of interest (Mairhofer 2010).

Amino acid auxotrophy has been used in another approach. A genomic deletion of the proBA gene is complemented by proBA on the expression plasmid comprising, in addition, the gene of interest (Fiedler 2001).

There are certain disadvantages of the aforementioned methods for example the intrinsic difficulties in the construction of the respective dapD, proBA or auxotrophic mutants and the need of defined culture media composition and even if the cultivation is done in a defined culture medium there is still the risk of cross feeding, as the result of secretion or cell-lysis, that supports growth of plasmid deficient cells, during fermentation.

A similar approach of complementation has been reported for the essential infA gene which is deleted in the genome and becomes complemented by a successfully transformed expression plasmid such that the gene of interest becomes expressed in the transformed bacteria (Hägg et al, J. Biotechnol., (2004) and its corresponding PCT-application WO2003/000881).

Further approaches are based on bacteria that are stably transformed with helper plasmids which express the essential infA gene and comprise a temperature sensitive origin of replication (ori) and a resistance gene for a first selection marker. Those bacteria are grown at a temperature allowing the ori to operate. After transformation with an expression plasmid having a normal ori and a further selection marker, successful transformed bacteria can be selected and grown at a normal temperature and in the presence of the selection agent for the selection marker of the expression plasmid (WO2017/097383). Bacteria that are treated by a heat shock, however, develop a stress response which is inferior for recombinant manufacture.

Moreover, thermo-sensitive approaches also use leader sequences for the infA. The endogenous infA expression is inhibited at a lower temperature by a leader sequence forming a hairpin structure at the 5′end of the transcript. The expression plasmid comprises in addition to the gene of interest an infA gene which can be transcribed and translated properly at lower temperatures. Accordingly, successfully transformed bacteria will grow and be viable at lower temperatures in such an approach while untransformed bacteria will die because the endogenous infA transcripts cannot be translated into infA protein (WO2018/083116). Bacteria can only be propagated at low temperatures which is inferior for recombinant manufacturing.

The abovementioned methods to stabilize plasmids in expression systems suffer from a couple of different disadvantages such as: (i) the requirement to create the respective expression strains in laborious time-consuming and expensive multi-stage process, which has to be repeated for each and every new product to be produced by said expression strains; (ii) the necessity to use during the final expression strain construction a couple of different plasmids containing selection markers such as antibiotic, toxin resistance genes or auxotrophic marker; (iii) the need to delete first the genomic copy of the respective essential gene after plasmid transformation containing said essential gene and a selection marker and second, if possible at all, to delete the selection marker from the plasmid; (iv) after construction of the production strain the risk of cross feeding during fermentation associated with the loss of the expression plasmids and a combined reduced production yield; (v) the risk of recombination of the essential gene from the plasmid into the genome and/or (vi) a low success rate in each process step during the creation of the final expression strain, which leads to on overall low efficiency in the strain construction.

However, it would be highly desirable to develop production bacterial strains expressing a desired gene of interest which are independent of complex and cumbersome cloning and the aforementioned mechanisms such as temperature shifts.

The technical problem underlying the present invention may be seen as the provision of means and methods for complying with the aforementioned needs. The technical problem is solved by the embodiments characterized in the claims and herein below.

Thus, the invention relates to a recombinant bacterial cell comprising in its genome at least one essential gene the endogenous promoter of which has been replaced by a heterologous expression control sequence being inducible by an inducer molecule, such that the expression of said essential gene in the recombinant bacterial cell is dependent on the presence of said inducer molecule.

It is to be understood that in the specification and in the claims, “a” or “an” can mean one or more, depending upon the context in which it is used. Thus, for example, reference to “an” item can mean that at least one item can be utilized.

As used in the following, the terms “have”, “comprise” or “include” are meant to have a non-limiting meaning or a limiting meaning. Thus, having a limiting meaning these terms may refer to a situation in which, besides the feature introduced by these terms, no other features are present in an embodiment described, i.e. the terms have a limiting meaning in the sense of “consisting of” or “essentially consisting of”. Having a non-limiting meaning, the terms refer to a situation where besides the feature introduced by these terms, one or more other features are present in an embodiment described.

Further, as used in the following, the terms “preferably”, “more preferably”, “most preferably”, “particularly”, “more particularly”, “typically”, and “more typically” or similar terms are used in conjunction with features in order to indicate that these features are preferred but not mandatory features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number. Depending on the item the term refers to the skilled person understands as to what upper limit the term may refer, if any.

The term “recombinant” as used herein refers to a bacterial cell which has been genetically modified. Preferably, said genetic modification may be a permanent modification. Accordingly, encompassed in accordance with the present invention are any modifications of the genome of the bacterial cell including those achieved by recombination techniques and those achieved by gene editing techniques. Modifications of the genome may be carried out, e.g., by using CRISPR-Cas-9 systems, lambda red recombination-based systems (the technology is also called Red/ET recombination or Red/ET recombineering), site-specific recombination techniques including, e.g., Cre-recombinase-based systems, FLP-FRT-based systems, transposon-based techniques or random integration techniques.

The term “bacterial cell” as used herein refers to a cell of gram-positive or gram-negative bacteria suitable for the recombinant manufacture of a compound of interest.

Depending on the compound of interest to be manufactured and the amount of compound of interest envisaged, the skilled person is well aware of what bacterial cell is suitable. Typically, the bacterial cell is selected from the group consisting of genera Escherichia, Vibrio, Bacillus, Lactobacillus, Acetobacter, Corynebacterium, Brevibacterium, Pseudomonas, Streptomyces, Gluconobacter, Clostridium, Streptococcus, Zooepidemicus, Basfia, Crytococcus, Rhodotorula, Nocardia, Erwinia, Xanthomonas, Leuconostoc and Klebsiella such as: Escherichia coli, Vibrio natriegens, Bacillus species, preferably, Bacillus subtilis, Bacillus licheniformis, Bacillus lentus, Bacillus amyloliquefaciens, Bacillus brevis, Bacillus stearothermophilus, Bacillus clausii, Bacillus alkalophilus, Bacillus coagulans, Bacillus circulans, Bacillus pumilus, Bacillus thuringiensis, Bacillus psuedofirmus, Bacillus marmarensis, Bacillus cellulolyticus, Bacillus hemicellulolyticus, Bacillus clarkia, Bacillus akibai, Bacillus gibsonii, Bacillus lautus, Bacillus megaterium or Bacillus halodurans, Lactobacillus species, preferably, Lactobacillus plantarum, Lactobacillus brevis, Lactobacillus coryniformis, Lactobacillus sanfranciscensis, Lactobacillus acidophilus, Lactobacillus delbrueckii, Lactobacillus helveticus, Lactobacillus curvatus or Lactobacillus varians, and Acetobacter species, preferably, Acetobacter aceti or Acetobacter pasteurianus. Corynebacterium species, preferably, Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola, Corynebacterium effiziens, Corynebacterium efficiens, Corynebacterium deserti, Brevibacterium flavum, Brevibacterium lactofermentum, Brevibacterium divarecatum, Pseudomonas putida, Pseudomonas syringae, Streptomyces species, preferably, Streptomyces coelicolor, Streptomyces lividans, Streptomyces albus, Streptomyces avermitilis, Gluconobacter oxydans, Gluconobacter morbifer, Gluconobacter thailandicus, Clostridium species, preferably, Clostridium acetobutylicum, Clostridium saccharobutylicum, Clostridium beijerinckii, Streptococcus species, preferably, Streptococcus aureus, Streptococcus equisimilis, Streptococcus pyogenes, Streptococcus uberis, Streptococcus equi subsp., Zooepidemicus, Basfia succiniciproducens, Cryptococcus laurentii, Rhodotorula glutensis, Nocardia mediterranei, Erwinia herbicola, Xanthomonas campestris, Leuconostoc mesenteroides and Klebsiella pneumoniae. Preferably, the bacterial cell referred to herein is a gram-negative bacterial cell. Preferably, said bacterial cell is a cell of the genus Escherichia such as an E. coli cell. Moreover, it is, preferably, envisaged that the bacterial genome lacks any gene being capable of functionally complementing the at least one essential gene in the absence of the inducer molecule.

The term “endogenous essential gene” as used herein refers to a gene which is naturally occurring in the genome of the bacterial cell and which is required for proper function of the bacterial cell. The absence of a functional endogenous essential gene as referred to herein shall, typically, result in lethality and/or growth arrest of the bacterial cell. Preferably, the endogenous essential gene is required for proper cell growth and/or viability of said bacterial cell.

The term “essential gene” as used herein refers to a gene, which is required for the survival of an organism, microorganism or a cell preferably a bacterial cell. For example, such essential genes are necessary for cell division (e.g., ftsL, ftsN, ftsZ or parC) or to maintain the cellular structure (e.g., dapA, dapB, rodA or rodZ), the central metabolism, the gene translation into proteins (e.g., era, rpsB, grpE, mopA or mopB) or the DNA replication of a cell (e.g., infA, infC, frr, dnaJ or dnaK), RNA transcription (e.g., nusG or trmA) or to support transport processes within, into or out of the cell (e.g., secY or msbA). By definition, if an essential gene is inactivated by for example deletion or knock-out the cell will die.

### Essential Genes can be Classified as:

(a) conditional essential genes, which means such genes are only essential under certain circumstances or growth conditions, i.e., genes leading to an auxotrophy or which are only inactivated at certain temperatures. For example, auxotrophy is the inability of an (micro)organism to synthesize a particular organic compound for its growth i.e., a sugar, fatty acid or amino acid required.
(b) absolute essential genes, which means that they are absolutely required for the cell to grow, proliferate and survive even under optimal growth conditions. Said essential genes are indispensable for organisms e.g., for DNA replication, RNA, protein synthesis, central metabolism, cell wall or membrane synthesis or reproduction;
(c) “redundant” essential genes, which means that alternative metabolic pathways within a cell leading to the same synthesis intermediate or final product often render essential genes non-essential, for example, if there are two enzymes within a cell synthesizing the same product the redundant enzyme can replace the other gene copy. If both genes are deleted and no product is produced the indispensability of said essential gene(s) become visible;
(d) a combination of essential genes, which means that said genes alone are non-essential however if another gene is inactive and therefore another gene deficiency is existing such gene becomes essential.

Preferably the term “essential gene” as used herein refers to at least one gene, which is an absolute essential gene according to (b) or it refers to at least one gene which is a combination of essential genes gene according to (d). Thus, in the bacterial cell of the invention, said at least one essential gene (i) is an absolute essential gene which is absolutely required for proper cell growth, proliferation and/or survival even under optimal growth conditions or which is indispensable for DNA replication, RNA synthesis, protein synthesis, central metabolism, cell wall or membrane synthesis or reproduction or (ii) is a combination of essential genes which are in combination absolutely required for proper cell growth, proliferation and/or survival even under optimal growth conditions or which is indispensable for DNA replication, RNA synthesis, protein synthesis, central metabolism, cell wall or membrane synthesis or reproduction.

More preferably, the at least one endogenous essential gene is selected from the group consisting of: infA, infC/IF-3, dnaJ, dnaK, era, frr, ftsL, ftsN, ftsZ, grpE, mopA, mopB, msbA, nusG, parC, rpsB, secY and trmA. More preferably such endogenous essential gene is selected from the group consisting of gens involved in the DNA replication of a cell, the RNA transcription or the support of transport processes within, into or out of the cell. In particular, it is selected from the group consisting of the following genes: infA, infC, frr, dnaJ, dnaK, nusG, trmA, secY and msbA. Most preferably, said at least one essential gene is infA, secY or frr.

Nucleic acid sequences of the aforementioned essential genes are known in the art for various bacterial species including those bacterial species explicitly mentioned elsewhere herein. Besides those nucleic acid sequences disclosed in the prior art, the aforementioned endogenous essential genes may also encompass variant sequences. Preferably, such a variant sequence still encodes a gene product which is capable to exert the biological function of the gene product of the essential gene. Typically, the variant nucleic acid sequence is at least 70%, at least 75%, at least 80%, at least 85%, at least 90%, at least 95%, at least 96%, at least 97%, at least 98% or at least 99% identical to the specific sequence for the endogenous essential gene disclosed in the prior art. Yet the endogenous essential genes may also encompass those which comprise nucleic acid sequences which encode gene products, i.e., proteins having an amino acid sequences which is at least 70%, at least 75%, at least 80%, at least 85%, at least 90%, at least 95%, at least 96%, at least 97%, at least 98% or at least 99% identical to the specific sequence for the gene product encoded by endogenous essential gene disclosed in the prior art. Nucleic acid sequence identity or amino acid sequence identity as referred to herein can be, preferably, determined by determining the number of identical amino acids between two nucleic acid sequences or amino acid sequences wherein the sequences are aligned so that the highest order match is obtained. It can be calculated using published techniques or methods codified in computer programs such as, for example, BLASTP, BLASTN or FASTA. The alignments for sequences are preferably made with the program “NEEDLE” [The European Molecular Biology Open Software Suite (EMBOSS)], which is based on the algorithm from Needleman and Wunsch [J. Mol. Biol. (1979) 48, p. 443-453] and using the default parameters (for example: gapopen=10.0, gap extend=0.5 and matrix 0 EDNAFULL)]. The percent sequence identity values are, typically, calculated over the entire nucleic acid or amino acid sequence or over a fragment of the nucleic acid or amino acid sequences being at least 50% in length of the entire sequences. For calculation of the percentage of sequence identity, typically, the default settings of the aforementioned computer programs are to be used. Preferred nucleic acid sequences for the open reading frames of the aforementioned essential genes from infA, secY andfrr. E. coli are also shown in SEQ ID NO: 15 (infA), SEQ ID NO: 16 (secY) and SEQ ID NO: 37 (frr), respectively.

Moreover, the promoter of the aforementioned essential gene is a region in the 5′ upstream non-coding region of the gene which comprises at least one expression control sequence naturally governing the expression of the essential gene. A promoter as referred to herein typically comprises binding sites for basal transcription factors such as sigma factor that are needed for recruiting RNA polymerase and initiating transcription of RNA from the gene. Moreover, the promoter also, preferably, comprises further transcription factor binding sites that are bound by transcription factors naturally required for proper transcription of the essential gene. Typically, the promoter of the essential gene is located within a DNA segment upstream of the transcription initiation site. Said DNA segment starting with the transcription initiation may, preferably, have a length of at most 100 bp, at most 200 bp, at most 300 bp, at most 400 bp, at most 500 bp, at most 750 bp or at most 1,000 bp further upstream. Promoter regions for the essential genes referred to above are well known to the skilled person.

The endogenous promoter of the at least one essential gene shall be replaced by a heterologous expression control sequence being inducible by an inducer molecule. As a consequence of said replacement the expression of the essential gene shall no longer be dependent on its endogenous promoter. Rather, the expression of the at least one essential gene shall become dependent as a consequence of the replacement on the heterologous expression control sequence being inducible by an inducer molecule and, thus, become dependent on the presence of said inducer molecule in said bacterial cell. It will be understood that the promoter may be replaced by, i.e. exchanging the DNA segment harboring the promoter by a polynucleotide harboring the heterologous expression control sequence. Yet, the endogenous promoter may also be inactivated and a polynucleotide comprising the heterologous expression control sequence may be introduced into the genome of the bacterial cell such that the expression of the at least one essential gene becomes dependent on the said heterologous expression control sequence. The inactivation of the endogenous promoter is typically the result of a genetic modification. In particular, it is envisaged that the inactivated endogenous promoter comprises at least one nucleotide deletion, substitution and/or addition compared to the wild-type version. The result of said genetic modification may be that the promoter is no longer capable of initiating transcription of the at least one essential gene. Preferably, the endogenous promoter in the genome of the bacterial system is inactivated by using CRISPR-Cas-9 systems, lambda red recombination-based systems, site-specific recombination techniques including, e.g., Cre-recombinase-based systems, FLP-FRT-based systems, transposon-based techniques or random integration techniques. Yet, the endogenous promoter may also be inactivated by other techniques such as random mutagenesis and selection for inactivated bacterial cells. For random mutagenesis, bacterial cells are treated with a mutagenizing agent or radiation in order to randomly introduce DNA modifications, such as substitutions or deletions. More preferably, replacement of the endogenous promoter may be carried out as described herein below by any of the methods of the invention or as described in the accompanying Examples, below.

It will be understood that the elements of the at least one essential gene other than the promoter, i.e. the coding sequence (structural gene) and the 3′elements of the gene. shall, preferably, remain at their endogenous location. Accordingly, the at least one essential gene in the bacterial cell of the invention having a replaced endogenous promoter as specified elsewhere herein shall, typically, possess the same neighboring sequence at least at the 3′end of the structural gene as the at least one essential gene in unmodified form.

The term “heterologous expression control sequence being inducible by an inducer molecule” as referred to herein relates to a nucleic acid sequence which is capable when being operatively linked to a coding nucleic acid sequence of a gene to govern the expression of said coding nucleic acid sequence of the gene. The expression control sequence is typically a promoter comprising transcription and translation regulating elements. The expression control sequence shall be inducible by an inducer molecule, i.e. its function shall depend on the presence and/or amount of an inducer molecule. Such inducer-dependent expression control sequences are known in the art and encompass, preferably, AraC/PBAD promoter, RhaR-RhaS/rhaBAD promoter, XylS/Pm promoter, NitR/PnitA promoter, and ChnR/Pb inducer/promoter (see, e.g., Brautaset 2009, Microb Biotechnol 2(1): 15-30). Other inducer-dependent systems may be a tetracycline-inducible promoter (tetON/OFF), a lac/IPTG promoter, an ethanol-inducible promoter (AlcA/AlcR), a steroid-inducible promoter (LexA/XVE) or a vanillate-inducible promoter. Moreover, the inducible expression control sequence referred to herein shall be heterologous with respect to the wild type, i.e. naturally occurring, bacterial cell, i.e. it shall be an expression control sequence which does not naturally occur in the recombinant bacterial cell or which is not natively associated with the endogenous essential gene in the said bacterial cell. In other words, the term “heterologous” shall mean with respect to the inducible expression nucleic acid sequence which is operably linked to, or is manipulated to become operably linked to, a second nucleic acid sequence, e.g., a promoter, a gene or operon to which it is not operably linked in nature, e.g., in the genome of a wildtype microorganism, or to which it is operably linked at a different location or position in nature, e.g., in the genome of a wildtype microorganism. Not “natively associated with” or not “operably linked” means that the recombinant bacterial cell (=“host cell”) does not naturally have this control sequence functionally linked to the coding nucleic acid sequence of the essential gene, which expression is controlled by the heterologous control sequence. Said inducible expression nucleic acid sequence might be originated from the same microbial systematic species or from a different species.

The term “inducer molecule” as referred to in accordance with the present invention relates to any molecule that is capable of acting as an inducer of expression of a gene being operatively linked to the heterologous expression control sequence being inducible by said inducer molecule. Typically, inducer molecules may be metabolic products which are metabolized by bacteria such as sugars, preferably, arabinose, rhamnose, xylose or sucrose, substituted benzenes, cyclohexanone-related compounds, F-caprolactam, propionate, thiostrepton, alkanes, isopropyl β-D-1-thiogalactopyranoside, tetracycline, anhydrotetracycline or peptides. Preferably, said inducer molecule is selected from the group consisting of: arabinose, rhamnose, xylose, sucrose, tetracycline, anhydrotetracycline and IPTG.

Advantageously, it has been found in accordance with the studies underlying the present invention that by replacing the endogenous promoter of an endogenous essential gene in the genome of a bacterial cell by an expression control sequence which can be controlled by an inducer molecule, a recombinant bacterial cell can be provided in which the viability and/or growth depends on the presence or amount of the inducer molecule in the culture. Such a recombinant bacterial cell can be used as a common platform for generating recombinant bacterial cells useful for the manufacture of a variety compounds of interest, i.e. protein producing bacterial cells. Thanks to the provision of the aforementioned recombinant bacterial cell as a common platform, it is no longer necessary to generate bacterial cells producing a desired compound of interest without the use of antibiotics in a cumbersome and inefficient manner. In particular, some prior art techniques require individual generation of the producing bacterial cells from scratch, i.e., the unmodified bacterial cells, using individual complementing plasmids for each case or, in the case of knock out approaches, the application of helper plasmids in order to remove resistance genes from the final producing bacterial cells. Thanks to the present invention a common bacterial cell can be used as a platform. A given expression plasmid used to generate a producing bacterial cell can be simply modified with less cloning efforts to comply with the system. There is no need for helper plasmids and for removing them, e.g., by using heat treatment steps above 37° C. culture temperature.

It will be understood that a producing bacterial cell for a desired compound of interest to be used in a recombinant manufacturing process can be generated from the aforementioned recombinant bacterial cell by introducing an expression plasmid into said recombinant bacterial cell.

Thus, in a preferred embodiment of the recombinant bacterial cell of the present invention, said bacterial cell comprises an expression plasmid which comprises at least one plasmid copy of the at least one essential gene, wherein said plasmid copy is under the control of the expression control sequence which is biologically active in said bacterial cell.

The aforementioned bacterial cell according to the present invention, the plasmid also is maintained because the cells are dependent on the plasmid-born copy of the at least one essential gene, e.g., infA gene, as used, inter alia, in the Examples, below. However, the present invention contains further critical improvements:
1) strain modification to enable essential gene-based plasmid maintenance is achieved using fewer steps and highly efficient lambda-red based recombineering resulting in a faster and more robust protocol that is feasible in a variety of strains backgrounds, included strains difficult to modify;
2) strain modification to enable essential gene-based plasmid maintenance only needs to be completed once per strain, rather than every time a new plasmid is used; and
3) the plasmid of interest can be transformed into the modified strain without requiring an antibiotic resistance marker, making the system completely independent of antibiotic resistance genes.

The expression plasmid might have at least one origin of replication (=“ori”) with either a broad-host range, which means the plasmid is able to replicate in a lot of taxonomically different species for example oriV, oriB or oriS, or with a narrow-host range, which means the plasmid is able to replicate in a few taxonomically different species or potentially only in one species for example the ColE1 ori. Broad-host range plasmids are for example RK2, RP4, RP1, R68, pUC1, pSa, pSFA231, pBC1, pWVO1, pLF1311, pAP1, pBBR1, pLS1, pIP501, pJD4, RSF1010/R1162/R300B or pPS10, which is conditional broad-host range. Some of the plasmids have one ori, which can initiate replication in one or more species or a couple of different ori(s), each of which can replicate in one or more species. Narrow-host range plasmids are for example pET, pBR322, pUC18, pUC19 or pMB1.

Expression plasmids, which can be used for the present invention are for example plasmids such as RK2, RP4, RP1, R68, pUC1, pSa, pSFA231, pPS10, pBC1, pEP2, pWVO01, pLF1311, pAP1, pBBR1, pLS1, pIP501, pJD4, pRSF1010/R1162/R300B, pPS10, pIP501, pRK290, pLAFR1, pLAFR5, pRS44, pJB653, pFAJ1700, pDSK509, pKT210, pAYC32, pAYC51/52, pJFF224-NX, pBHR1, pGEX, pSC101, p15A, pJF118EH, pKK223-3, pUC18, pUC19, pBR322, pACYC184, pASK-IBA3, pET and any expression plasmid created or derived therefrom.

Depending on the at least one further nucleic acid of interest (definition see below) within the plasmid the skilled person knows, which plasmid to choose for an optimal expression of said nucleic acid of interest and the best production of said nucleic acid or the product derived therefrom. Some of the plasmids mentioned herein have high copy numbers within the cell for example pUC18, pMBI or RSF1030 others have lower copy numbers for example pBR322, pSC101 or p15A or potentially only one copy within the cell for example F1 or P1.

Preferably, said expression plasmid is selected from the group consisting of: pJF118EH, pKK223-3, pUC18, pBR322, pACYC184, pASK-IBA3, pET and any expression plasmid related or derived therefrom. It will be understood that although some plasmids may comprise selection markers such as antibiotic resistance genes, those genes are not required for using the recombinant bacterial cell according to the invention. Rather, the expression of the plasmid copy of the at least one essential gene shall be sufficient for selecting those recombinant bacterial cells which have successfully taken up the expression plasmid. More preferably, the expression plasmid may be a plasmid as described in the accompanying Examples, below. It is envisaged in accordance with the present invention that the expression of the plasmid copy of the at least one essential gene is governed by an expression control sequence which is biological active under physiological conditions in the recombinant bacterial cell. Thus, said expression control sequence may be a constitutively active promoter which is permanently expressed in the cell. Alternatively, it may be a promoter which is constitutively expressed during the phase of growth in the recombinant bacterial cell. Suitable expression control sequences are well known to the person skilled in the art. They include, e.g., the constitutively active promoters from E. coli described in Shimada et al. 2014, PLoS ONE 9(3): e90447. https://doi.org/10.1371/journal.pone.0090447. Similar constitutively active promoters can be found for other bacteria and have been reported in the prior art already. Preferably, said expression control sequence which is biologically active is the expression control sequence which is natively associated with the at least one endogenous essential gene. Thus, more preferably, the said expression control sequence is a promoter selected from the group consisting of infA promoter, infAp2 promoter, infC/IF-3 promoter, dnaJ promoter, dnaK promoter, era promoter, frr promoter, ftsL promoter, ftsN promoter, ftsZ promoter, grpE promoter, mopA promoter, mopB promoter, msbA promoter, nusG promoter, parC promoter, rpsB promoter, secY promoter and trmA promoter. Most preferably, said at least one essential gene is infA promoter.

Preferably, said expression plasmid comprises at least one further nucleic acid of interest which is required for the recombinant manufacture of a compound of interest in said recombinant bacterial cell.

The term “at least one further nucleic acid of interest” as used herein relates to a nucleic acid which encodes a gene product, i.e. a protein, peptide or RNA, which is required to manufacture a compound of interest envisaged to be produced by the recombinant bacterial cell of the invention. It will be understood that the compound of interest to be manufactured may also be the encoded gene product itself. Typically, however, the gene product shall be required for the recombinant manufacture of the compound of interest. Preferably, a protein as referred to herein may be an enzyme which is required for one or more catalytic conversions required during the recombinant manufacturing process. Alternatively, it may be a binding protein, such as a chaperone, which binds to and protects a compound of interest and, thus, may facilitate purification of said compound. A peptide referred to herein may be useful for growth control of the bacterial cells within the culture or may be useful as an antimicrobial peptide if the peptide itself is envisaged to be produced as a compound of interest. An RNA as referred to herein may be an inhibitory RNA, such as siRNA or microRNA, or an enzymatically active RNA. Inhibitory RNAs or ribozymes are both suitable agents for controlling the presence and or amount of certain other RNAs such as mRNAs encoding certain proteins or peptides. By controlling such mRNAs, the inhibitory RNAs or ribozymes may control the presence or amount of biological activity of a protein present in the bacterial cell, e.g., the presence or amount of enzymatic activity. It will be understood that a recombinant manufacturing process can, hence, be controlled by those agents as well.

It will be understood that the nucleic acid of interest required for the recombinant manufacture of the compound of interest shall be operatively linked to an expression control sequence which is biologically active in the bacterial cell of the present invention or which can be activated during culture. Accordingly, such an expression control sequence may comprise a constitutively active promoter as well as an inducible promoter. Constitutively active promoters have been described elsewhere herein already. It will be understood that an inducible promoter used for governing expression of the nucleic acid of interest shall not be controlled by the inducible molecule used for controlling the expression of the at least one introduced copy of the said at least one essential gene according to the present invention. Moreover, it is to be understood that if an inducible promoter is used for producing the compound of interest, the said promoter must be induced, i.e. the inducing stimulus must be applied to the recombinant bacterial cell culture.

Typical inducing stimuli applied in this context may be altered physical conditions, e.g., the application of a heat stimulus (also called “heat shock”) or the administration of an inducing molecule. Inducible promoters, typically, encompass heat-inducible promoters as well as compound-inducible promoters such as those referred to elsewhere herein. The skilled artisan is well aware of which promoters may be used in this context.

Care should be taken that the expression plasmid, in principle, allows for expression of both, the at least one plasmid copy of the at least one essential gene as referred to above and the at least one further nucleic acid of interest as specified above. Thus, both expression cassettes need to be arranged in the expression plasmid such that there is no interference between the expression of either expression cassette by the other one.

The term “compound of interest” as used in accordance with the present invention refers to any compound which can be recombinantly manufactured in the recombinant bacterial cell of the present invention. The compound of interest shall be, typically, manufactured due to the expression and/or consecutive reaction(s) of the gene product encoded by the further nucleic acid of the expression plasmid in the producing bacterial cells described herein before. Preferably, the compound of interest belongs into a chemical class selected from the group consisting of: lipids, amino acids, purines including nucleotides and nucleosides based thereon, pyrimidines including nucleotides and nucleosides based thereon, isoprenoids, proteins, peptides, nucleic acids, terpenes, carotenoids, saccharides, polysaccharides, alkaloids, alcohols, antibiotics, vitamins, polyhydroxyalkanoates, polyamides, polylactic acids, enzymes, coenzymes, organic acids, and the like. Yet, the compound of interest may also be the gene product itself encoded by the further nucleic acid of interest comprised in the expression plasmid. Typically, the compound of interest in such a case is a protein, peptide or RNA molecule. Moreover, the expression plasmid itself or a part thereof, i.e. a nucleic acid sequence such as DNA or RNA molecule, may be envisaged as a compound of interest manufactured by the producing recombinant bacterial cell of the present invention.

Preferably, nucleic acid of interested as referred to herein may be a therapeutic nucleic acid such as unmodified DNA or RNA or closely related compounds used for treating diseases. Typically, a plasmid DNA as a nucleic acid of interest according to the invention may be used for preparing a pharmaceutical composition or vaccine or for use in gene therapy. For these applications, it is essential that no antibiotic resistance marker genes are present on the plasmid DNA. Since 1989, more than 3,000 clinical trials have been reported, and approximately 15% of them use plasmid DNA (pDNA) as the vector system (Alves et al. 2021). Compared to viral and RNA-based vectors, plasmid DNA has lower toxicity, higher stability, and is easier and cheaper to produce. However, pDNA vectors have some limitations when compared with viral vectors, namely lower transfection efficiency and adequate expression over time. The transfection efficiency is improved by decreasing the size of the pDNA vector (Oliveira and Mairhofer 2013). Loss of expression over time, or gene silencing, has recently been shown to occur when there is >1 kb of bacterial DNA in the pDNA vector (Lu et al. 2012). In addition, the presence of unmethylated CpG motifs commonly found in prokaryotic DNA can trigger immune responses (Alves et al. 2021), further highlighting the importance of minimizing pDNA vector size. A small plasmid backbone is therefore critical for therapeutic applications.

The total length of a plasmid backbone consisting of, e.g., the infA gene and a p15A origin of replication is about 1 kb. The total length of a plasmid backbone consisting of, e.g., the infA gene and an R6K origin of replication is about 800 bp in size. Thus, the plasmid maintenance system described elsewhere herein is, preferably, well-suited for the production of pDNA for gene therapy and other therapeutic applications.

Advantageously, it has been further found in accordance with the studies underlying the present invention that using a recombinant bacterial cell comprising in its genome at least one essential gene the endogenous promoter of which has been replaced by a heterologous expression control sequence being inducible by an inducer molecule as a platform, various different producing bacterial cells can be easily generated that can be applied for the recombinant manufacture of a desired compound of interest. Said platform bacterial cells can be cultivated in the presence of the inducer molecule. After transformation with an expression plasmid as described herein, the transformed bacterial cells will no longer be cultured in the presence of the inducer molecule and only those bacteria will grow and/or be viable which have taken up the expression plasmid which allows for expression of the at least one essential gene. There is no antibiotic selection marker and no cultivation under selective conditions necessary since selection of the transformed bacteria takes place under physiological conditions once the inducer molecule is withdrawn from the culture. Thus, the recombinant bacterial cells of the present invention avoid the use of antibiotics which are required in conventional recombinant manufacturing process for selection and cultivation. Neither the expression plasmid which is introduced, nor the platform bacterial cell contain an antibiotic resistance marker gene. The expression plasmid in a producing bacterial cell is kept stable in the cell even under high density fermentation conditions and under optimal and suboptimal culture temperatures and conditions.

The present invention also relates to a method for generating the recombinant bacterial cell of the invention comprising the steps of.
(a) exchanging in the genome of a bacterial cell the endogenous promoter of at least one essential gene by a first polynucleotide comprising a selection cassette comprising a promoter that can be active in the bacterial cell operatively linked to
   (i) a first selection gene that confers sensitivity for a first selection agent such that the bacterial cell will not grow and/or will die in the presence of said first selection agent, and
   (ii) a second selection gene that confers resistance for a second selecting agent such that the bacterial cell will be able to grow in the presence of said second selecting agent,
   such that the expression of the at least one essential gene, the first and the second selection gene will become dependent on the said promoter;
(b) selecting a bacterial cell which has integrated into its genome said first polynucleotide using the second selecting agent;
(c) exchanging said first polynucleotide which has been integrated into the genome by a second polynucleotide comprising a heterologous expression control sequence being inducible by an inducer molecule such that the expression of the at least one essential gene becomes dependent on said heterologous expression sequence; and
(d) selecting a bacterial cell which has integrated into its genome said second polynucleotide using the first selection molecule and the inducer molecule.

The method according to the present invention may consist of the aforementioned steps (a) to (d) or may comprise further steps, such as cultivating and/or pretreating the bacterial cell prior to step (a) and/or cultivating and/or treating the bacterial cell after step (d).

The term “promoter that can be active in the bacterial cell” refers to expression control sequences that govern expression of a gene operatively linked thereto in the bacteria cell. Said promoter may be an endogenous promoter that naturally occurs in the genome of the bacterial cell or it may be a heterologous promoter, i.e. a promoter from another species known to operate in the bacterial cell. Moreover, said promoter, preferably, can be active in the bacterial cell as a constitutively active or an inducible promoter. The skilled artisan is well aware of suitable promoters. More preferably, said promoter being constitutively active in the bacterial cell is selected from the group consisting of rpsL promoter, infA promoter, infAp2 promoter, em7 promoter, and cat promoter. Most preferably, the said promoter is the rpsL promoter. Also more preferably, said inducible promoter is selected from the group consisting of a heat-inducible promoter, tetracycline-inducible promoter, a lacI/IPTG promoter, an ethanol-inducible promoter (AlcA/AlcR), a steroid-inducible promoter (LexA/XVE), and a vanillate-inducible promoter. If an inducible promoter is used as promoter that can be active in the bacterial cell in accordance with the invention, it will be understood that said promoter is, preferably, different from the heterologous expression control sequence being inducible by an inducer molecule. In particular, the inducer for the inducible promoter and the inducer molecule controlling the expression of the at least one essential gene shall be different.

The term “operatively linked” as used herein means that two genetic elements referred to herein are functionally linked to each other. For example, an expression control sequence may be functionally linked to the coding sequence of a gene of interest if it is capable of governing its expression. Typically, the expression sequence may be positioned in physical proximity to the coding sequence of the gene, i.e. at its 5′end. However, it is also possible to position an expression control sequence at a certain distance to the coding sequence as long as it is still capable to govern the expression thereof. The skilled person is well aware of how such operative linkage can be achieved depending on a given expression control sequence.

The term “first selection gene” refers to a gene that encodes a gene product the expression of which in the bacterial cell confers sensitivity to the bacterial cell for a first selection agent. The first selection gene may, typically, encode a protein that when expressed in the bacterial cell will in the presence of the first selection agent either inhibit growth of said bacterial cell and/or be detrimental for viability of said bacterial cell potentially lethal. Suitable proteins may be enzymes, ribosomal proteins and other proteins required for translation, transcription factors, cell cycle proteins, and the like. Preferably, a first selection gene as referred to in accordance with the present invention is selected from the group consisting of: ribosomal S12 protein gene (rpsL), levansucrase (sacB), tRNA synthetase for phenylalanine (pheS), tetR, galactokinase (galK), thymidilate synthetase (thyA), and tolC. More preferably, the first selection gene is rpsL.

The term “first selection agent” as used herein refers to a compound that will be detrimental for viability and/or growth of a bacterial cell expressing a first selection gene that confers sensitivity for a first selection agent. Preferably, the first selection agent is selected from the group consisting of: streptomycin, sucrose, chloro-phenylalanine, chlortetracycline and fusaric acid, galactose, trimethroprim and derivatives thereof, and or example bacteriocins such as colicins like pore forming colicins, e.g., colicin A, B, E1, Ia, Ib, N or there like; colicin D, which is a translation factor inhibitor; colicin M, which blocks peptidoglycan synthesis or colicin E3 or E3, which have a nuclease activity, preferably colicin E1. More preferably, the first selection agent is streptomycin.

It is well known for which of the aforementioned first selection agent which first selection gene confers sensitivity. However, in the following table, preferred pairs are listed.

TABLE 1
First selection genes and first selection agents

| First selection gene | First selection agent |
|---|---|
| rpsL | streptomycin |
| sacB | sucrose |
| pheS | chloro-phenylalanine |
| tetR | chlortetracycline and fusaric acid |
| galK | galactose |
| thyA | trimethoprim and derivatives thereof |
| tolC | colicin E1 |

The term “second selection gene” refers to a gene that encodes a gene product the expression of which in the bacterial cell confers resistance to the bacterial cell for a second selection agent. The second selection gene may, typically, encode a protein that when expressed in the bacterial cell will in the presence of the second selection agent allow the said bacterial cell to survive and grow. In the absence of the protein encoded by the second selection gene, the bacterial cell will die or be growth inhibited in the presence of the said second selection agent. Suitable proteins may be enzymes, transporter protein, and the like. Preferably, a second selection gene as referred to in accordance with the present invention is selected from the group consisting of kmR, zeoR, ampR, tetR, cmR, hygR, specR, and BSD. More preferably, the second selection gene is kmR or zeoR. However, the skilled person is well aware that other resistance genes preferably antibiotic resistance genes can be used in similar way.

The term “second selection agent” as used herein refers to a compound that will be detrimental for viability and/or growth of a bacterial cell that does not express the second selection gene that confers resistance to the second selection agent. Preferably, the second selection agent is selected from the group consisting of kanamycin, zeocin, ampicillin, tetracycline, chloramphenicol, hygromycin, spectinomycin, and blasticidin. More preferably, the second selection agent is kanamycin or zeocin. However, the skilled person is well aware that other resistance genes preferably antibiotic resistance genes can be used in similar way. Furthermore, the skilled person is well aware that during the process as described the first and the second selection agent must be different from one another.

It is well known for which of the aforementioned first selection agent which first selection gene confers sensitivity. However, in the following table, preferred pairs are listed.

TABLE 2
Second selection genes and second selection agents

| Second selection gene | Second selection agent |
|---|---|
| kmR | kanamycin |
| zeoR | zeocin |
| ampR | ampicillin |
| tetR | tetracycline |
| cmR | chloramphenicol |
| hygR | hygromycin |
| specR | spectinomycin |
| BSD | blasticidin |

The term “exchanging” as used herein refers to any techniques which result in replacing the endogenous promoter of the at least one essential gene by a first polynucleotide comprising a selection cassette comprising a promoter that can be active in the bacterial cell operatively linked to (i) a first selection gene that confers sensitivity for a first selection agent such that the bacterial cell will not grow and/or will die in the presence of said first selection agent, and (ii) a second selection gene that confers resistance for a second selecting agent such that the bacterial cell will grow in the presence of said second selecting agent, such that the expression of the at least one essential gene, the first and the second selection gene will become dependent on the said promoter. Preferably, such exchange can be carried out by using site-specific or random recombination. Thus, it will be understood that the first polynucleotide may comprise upstream of the promoter that can be active in the bacterial cell and downstream of the second selection gene that confers resistance for a second selecting agent sequences of the bacterial genome that are naturally present upstream and downstream of the endogenous promoter of the at least one essential gene. The said first polynucleotide is typically integrated into the genome of the bacterial cell by using CRISPR-Cas-9 systems, lambda red recombination, site-specific or random recombination techniques as described elsewhere herein in more detail and, in particular, lambda red recombination (also called Red/ET recombination or Red/ET recombineering). How to carry out such recombination techniques is well known to the skilled artisan.

It will be understood that the viability and/or growth of the recombinant bacterial cell after having carried out the aforementioned step (a) of the method of the present invention will become dependent on the expression of the second selection gene that confers resistance for the second selecting agent such that the bacterial cell will grow and/or be viable in the presence of said second selecting agent.

By cultivating the recombinant bacterial cell in the presence of the second selection agent in step (b), bacterial cells can be selected that have properly integrated the first polynucleotide into their genome. Said selection is typically carried out by cultivating the bacterial cell in the presence of the selection agent for a time and under conditions which allow for selection.

Typical conditions and time are described in the accompanying Examples and can be determined by the skilled person without further ado.

In step (c), the first polynucleotide which has been integrated into the genome will be exchanged by a second polynucleotide comprising a heterologous expression control sequence being inducible by an inducer molecule such that the expression of the at least one essential gene becomes dependent on said heterologous expression sequence.

By cultivating the recombinant bacterial cell in the presence of the inducer molecule and the first selection agent, bacterial cells which have properly integrated into their genome said second polynucleotide can be selected. The first selection agent in this context will interfere with viability and/or growth of bacterial cells that have not exchanged the first by the second polynucleotide and, thus, still express the first selection gene that confers sensitivity for a first selection agent.

Preferably, said selection cassette is further comprising a restriction enzyme cleavage site at the 3′end of the second selection gene. Preferably, by treating the cells with a restriction enzyme that cleaves the first polynucleotide at the cleavage site at the 3′end of the second selection gene, selection of bacterial cells having properly integrated the second polynucleotide can be further improved. Therefore, the method of the invention further comprises after step (c) the step of treating the bacterial cell with the restriction enzyme such that a genome of a bacterial cell still comprising the first polynucleotide comprising the selection cassette will be cleaved between the second selection gene and the at least one essential gene while the genome of a bacterial cell having exchanged the first polynucleotide comprising a selection cassette which has been integrated into the genome by a second polynucleotide comprising a heterologous expression control sequence being inducible by an inducer molecule such that the expression of the at least one essential gene becomes dependent on said heterologous expression sequence remains unaffected by the restriction enzyme. More preferably, said restriction enzyme is selected from the group consisting of: I-SceI, I-CeuI, I-PpoI, PI-SceI, and PI-PspI. More preferably, the restriction enzyme is I-SceI.

Preferably, the bacterial cell is cultivated in step (d) and afterwards in the presence of the inducer molecule. By cultivating the recombinant bacterial cell in the presence of the inducer molecule, cell cultures of the recombinant bacterial cells can be generated which can be used as a common platform for the manufacture of various compounds of interest. Depending on the desired compound of interest, the recombinant bacterial cells of the said cultures may be individually transformed with an expression plasmid comprising at least one plasmid copy of the at least one essential gene, wherein said plasmid copy is under the control of the expression control sequence which is biologically active in said bacterial cell and wherein said expression plasmid comprises a nucleic acid of interest which is required for the manufacture of the compound of interest. Successful transformants will grow in the absence of the inducer molecule while untransformed bacterial cells will require the presence of the inducer molecule for growth and/or survival.

Particular preferred details for carrying of the aforementioned method are to be found in the accompanying Examples, below.

The invention also encompasses a method for generating the recombinant bacterial cell of the present invention comprising the steps of:
(a) exchanging in the genome of a bacterial cell the endogenous promoter of at least one essential gene by a polynucleotide comprising
   (i) a selection gene that confers resistance for a selection agent, said selection gene being flanked by recognition sites for site specific recombination in the bacterial cell, and
   (ii) a heterologous expression control sequence being inducible by an inducer molecule,
   such that the expression of the at least one essential gene becomes dependent on said heterologous expression control sequence and such that the bacterial cell will grow in the presence of said inducer molecule and in the presence of said selection agent;
(b) selecting a bacterial cell which has integrated into its genome said polynucleotide using the selection agent and the inducer molecule;
(c) removing the selection gene by using a recombinase that is capable of carrying out site specific recombination using the recognition sites flanking the selection gene such that the expression of the at least one essential gene remains dependent on said heterologous expression control sequence; and
(d) selecting a bacterial cell which has integrated into its genome said polynucleotide lacking the selection gene using the inducer molecule.

The term “selection gene” refers to a gene that encodes a gene product the expression of which in the bacterial cell confers resistance to the bacterial cell for a selection agent. The selection gene may, typically, encode a protein that when expressed in the bacterial cell will in the presence of the second selection agent allow the said bacterial cell to survive and grow. In the absence of the protein encoded by the second selection gene, the bacterial cell will die or be growth inhibited in the presence of the said selection agent. Suitable proteins may be enzymes, transporter protein, and the like. Preferably, said selection gene in the aforementioned method of the invention is selected from the group consisting of ampR, kmR, zeoR, tetR, cmR, hygR, specR, and BSD. More preferably the selection gene in the aforementioned method is ampR. The skilled person knows that other selection genes for antibiotics, toxins or other killing agents can be used in similar way.

The term “selection agent” as used herein refers to a compound that will be detrimental for viability and/or growth of a bacterial cell that does not express the selection gene that confers resistance to the selection agent. Preferably, said selection agent in the aforementioned method of the invention is selected from the group consisting of ampicillin, kanamycin, zeocin, tetracycline, chloramphenicol, hygromycin, spectinomycin, and blasticidin. More preferably, the selection agent in the aforementioned method is ampicillin.

The term “recognition sites for site specific recombination” as used herein refers to DNA sequences that are recognized specifically by enzymes that are capable of mediating site specific recombination. Such enzymes are typically recombinases such as Cre-recombinase-based systems (Cre/loxP), Dre-recombinase-based systems (Dre/rox), FLP-FRT-based systems or transposon-based techniques. Typically, such recognition sites shall be located in the DNA segments flanking the DNA segment to be affected by the recombination event and, thus, are marking the DNA region affected by recombination. Preferably, said recognition sites for site specific recombination in the bacterial cell are FRT sequences. Also preferably, said recombinase is a flippase (FLP) recombinase. Alternatively, the recognition sites for site specific recombination in the bacterial cell are, preferably, loxP sequences and the recombinase, preferably, is a Cre recombinase. The skilled person is well aware of how to use recognition sites for site specific recombination together with suitable recombinases in the method of the invention.

In step (a) of the aforementioned method of the invention, the endogenous promoter of at least one essential gene in the genome of a bacterial cell will be exchanged by a polynucleotide comprising (i) a selection gene that confers resistance for a selection agent, said selection gene being flanked by recognition sites for site specific recombination in the bacterial cell, and (ii) a heterologous expression control sequence being inducible by an inducer molecule. Preferably, such exchange can be carried out by using lambda red recombination (also called Red/ET recombination or Red/ET recombineering) or CRISPR-Cas-9 systems. It will be understood that if site specific recombination is used for the exchange in step (a), recognition sites, preferably, being different from those present in the polynucleotide referred to in step (a) and a recombinase being different from the recombinase used in step (c) shall be used. How to carry out such recombination techniques is well known to the skilled artisan. As a result, of a successful exchange as carried out in step (a), the expression of the at least one essential gene shall become dependent on the heterologous expression control sequence and the bacterial cell shall grow in the presence of the inducer molecule and in the presence of the selection agent.

Thus, in step (b) of the method, a bacterial cell which has integrated into its genome said polynucleotide will be selected using the selection agent and the inducer molecule. Said selection is typically carried out by cultivating the bacterial cell in the presence of the selection agent and the inducer molecule for a time and under conditions which allow for selection. Typical conditions and time are described in the accompanying Examples and can be determined by the skilled person without further ado. As a result of the removal of the selection gene, that the expression of the at least one essential gene shall remain dependent on said heterologous expression control sequence.

After selection of bacterial cells having properly integrated the polynucleotide as described above, a segment of the polynucleotide comprising the selection gene is removed in step (c) of the method by using a recombinase that is capable of carrying out site specific recombination using the recognition sites flanking the selection gene. As a result of the removal of the selection gene, the expression of the at least one essential gene shall remain dependent on the heterologous expression control sequence.

Since the heterologous expression control sequence is inducible by the inducer molecule, a bacterial cell which has integrated into its genome the polynucleotide lacking the selection gene can be selected in step (d) using the inducer molecule. Said selection is typically carried out by cultivating the bacterial cell in the presence of the inducer molecule for a time and under conditions which allow for selection. Typical conditions and time are described in the accompanying Examples and can be determined by the skilled person without further ado.

Particular preferred details for carrying of the aforementioned method are to be found in the accompanying Examples, below.

Yet, the invention relates to a method for generating the recombinant bacterial cell of the invention comprising the steps of:
(a) exchanging in the genome of a bacterial cell the endogenous promoter of at least one essential gene by a polynucleotide comprising
   (i) a heterologous expression control sequence being inducible by an inducer molecule,
   (ii) a promoter that can be active in the bacterial cell, and
   (iii) a selection gene that confers resistance for a selection agent, wherein a segment of the polynucleotide comprising (ii) the promoter that can be active in the bacterial cell and (iii) the selection gene but not the heterologous expression control sequence is flanked by recognition sites for site specific recombination in the bacterial cell,
      such that the expression of the at least one essential gene will become dependent on the promoter that can be active in the bacterial cell;
(b) selecting a bacterial cell which has integrated into its genome said polynucleotide using the selection agent;
(c) removing the segment of the polynucleotide comprising the promoter that can be active in the bacterial cell and the selection gene by using a recombinase that is capable of carrying out site specific recombination using the recognition sites flanking the segment of the polynucleotide such that the expression of the at least one essential gene becomes dependent on said heterologous expression control sequence; and
(d) selecting a bacterial cell which has integrated into its genome said polynucleotide lacking the segment flanked by the recognition sites using the inducer molecule.

Preferably, said selection gene in the aforementioned method of the invention is selected from the group consisting of: kmR, ampR, zeoR, tetR, cmR, hygR, specR, and BSD. More preferably the selection gene in the aforementioned method is kmR.

Preferably, said selection agent in the aforementioned method of the invention is selected from the group consisting of kanamycin, ampicillin, zeocin, tetracycline, chloramphenicol, hygromycin, spectinomycin, and blasticidin. More preferably, the selection agent in the aforementioned method is kanamycin.

In step (a) of the aforementioned method of the invention, the endogenous promoter of at least one essential gene by a polynucleotide comprising (i) a heterologous expression control sequence being inducible by an inducer molecule, (ii) a promoter that can be active in the bacterial cell, and (iii) a selection gene that confers resistance for a selection agent. A segment of the polynucleotide comprising (ii) the promoter that can be active in the bacterial cell and (iii) the selection gene but not the heterologous expression control sequence is flanked by recognition sites for site specific recombination in the bacterial cell. Preferably, the exchange in step (a) of the method can be carried out by using lambda red recombination (also called Red/ET recombination or Red/ET recombineering) or CRISPR-Cas-9 systems. It will be understood that if site specific recombination is used for the exchange in step (a), recognition sites, preferably, being different from those present in the polynucleotide referred to in step (a) and a recombinase being different from the recombinase used in step (c) shall be used. How to carry out such recombination techniques is well known to the skilled artisan. As a result, of a successful exchange as carried out in step (a), the expression of the at least one essential gene in the bacterial cell will become dependent on the promoter that can be active in the bacterial cell.

Thus, in step (b) of the method, a bacterial cell which has integrated into its genome said polynucleotide will be selected using the selection agent. Said selection is typically carried out by cultivating the bacterial cell in the presence of the selection agent for a time and under conditions which allow for selection. Typical conditions and time are described in the accompanying Examples and can be determined by the skilled person without further ado.

After selection of bacterial cells having properly integrated the polynucleotide as described above, a segment of the polynucleotide comprising the selection gene and the promoter that can be active in the bacterial cell is removed in step (c) of the method by using a recombinase that is capable of carrying out site specific recombination using the recognition sites flanking the said segment. As a result of the removal of the selection gene and the promoter that can be active in the bacterial cell (i.e. the segment of the polynucleotide flanked by the recognition sites), the expression of the at least one essential gene shall become dependent on the heterologous expression control sequence.

Since the heterologous expression control sequence is inducible by the inducer molecule, a bacterial cell which has integrated into its genome the polynucleotide lacking the segment flanked by the recognition sites can be selected in step (d) using the inducer molecule. Said selection is typically carried out by cultivating the bacterial cell in the presence of the inducer molecule for a time and under conditions which allow for selection. Typical conditions and time are described in the accompanying Examples and can be determined by the skilled person without further ado.

Particular preferred details for carrying out the aforementioned method are to be found in the accompanying Examples, below.

The present invention also relates to a method for recombinant manufacture of a compound of interest comprising the steps of:
(a) introducing into a recombinant bacterial cell of the present invention an expression plasmid which comprises at least one plasmid copy of the at least one essential gene, wherein said plasmid copy is under the control of the expression control sequence which is biologically active in said bacterial cell; and
(b) cultivating the recombinant bacterial cell obtained in step a) in the absence of the inducer molecule.

The term “expression control sequence which is biologically active in said bacterial cell” refers to any promoter which naturally occurs in the bacterial cell or any heterologous or artificial promoter that is biologically active in said cell. Preferably, said expression control sequence which is biologically active in the bacterial cell can be a promoter that is biologically active in the bacterial cell as defined elsewhere herein. Alternatively, the said expression control sequence may be a promoter of an essential gene as referred to herein, such as, the infA, secY or frr promoter or any other promotor of an essential gene as disclosed herein.

Preferably, said expression control sequence which is biologically active is the expression control sequence which is natively associated with the at least one endogenous essential gene. Details are to be found elsewhere herein.

Preferably, said expression plasmid comprises at least one further nucleic acid of interest which is required for the recombinant manufacture of a compound of interest in said recombinant bacterial cell.

In step (a) of the aforementioned method, an expression plasmid which comprises at least one plasmid copy of the at least one essential gene, wherein said plasmid copy is under the control of the expression control sequence which is biologically active in said bacterial cell is introduced into the said cell. Typically, the expression plasmid is to be introduced by any conventional transformation method such as electroporation, TSS-, CaCl- or RbCl-transformation techniques or virus-mediated transduction. The skilled artisan is well aware of how such transformation techniques may be carried out.

Once the expression plasmid has been introduced, e.g., by any of the aforementioned transformation techniques, the recombinant bacterial cells are cultivated in step (b) of the aforementioned method of the invention in a suitable culture medium without antibiotics and under suitable culture conditions. In particular, the culture medium shall be free of the inducer molecule in order to achieve selection of those recombinant bacterial cells which have successfully taken up the expression plasmid. Moreover, the culture conditions shall, typically, allow for the recombinant manufacture of the compound of interest by the cultivated recombinant bacterial cells. As described elsewhere herein, this may require applying an expression stimulus to said cultured cells. However, the compound of interest may also be constitutively manufactured in the recombinant bacterial cells. Preferably, the cultivation is done until a desired level of the compound of interest is produced by the recombinant bacterial cells in the culture and the said cells, and afterwards, the cells and/or the culture medium used for cultivating the cells are harvested for obtaining the compound of interest. A particular preferred technique is described in the Examples, below.

The cultivation of the recombinant bacterial cells may also comprise several cultivation steps. For example, cultivation may comprise cultivating the recombinant bacterial cell in a pre-culture until the cells are exponentially growing and subsequently inoculating a culture in a production vial such as a bioreactor with said pre-culture in order to avoid that the cells in the bioreactor culture are in a lag stage or to at least reduce said lag phase in the bioreactor.

Such cultivation steps may include, without being limited to, the cultivation of the bacterial cells on a solid growth medium or the growth in liquid medium. Such medium and/or culture may be (i) a minimal medium that contains the minimal necessities for growth of the wild-type organism (e.g. containing only nutrients essential for growth of the bacterial cells such as water, an N- and/or C-source, vitamins, and various salts that contain essential elements required for protein and nucleic acid synthesis); (ii) a defined medium that is in its composition completely understood but in general does not contain any complex nutrition composition such as yeast extract or (iii) a complex medium with an undefined assortment and quantity of nutrients such as yeast extract, peptones from plants or animals, tryptones, corn steep liquor or similar protein sources. Either medium can be used for the expression of genes according to the current invention.

The culture medium as referred to herein is preferably a solution which comprises compounds that are required for proper growth and viability of the recombinant bacterial cells of the present invention. Typically, a culture medium as referred to herein comprises compounds which serve as a nitrogen and/or carbon source for the bacterial cells. Said compounds may be chemically defined compounds mixed together or obtained as a chemically undefined mixture from biological sources. Chemically defined compounds useful for culture media may be carbohydrates, organic acids, hydrocarbons, alcohols or mixtures thereof. Preferred carbohydrates are selected from the group consisting of glucose, fructose, sucrose, galactose, xylose, arabinose, sucrose, maltose, maltotriose, lactose, dextrin, maltodextrins, starch and inulin, and mixtures thereof. Preferred alcohols are selected from the group consisting of glycerol, methanol and ethanol, inositol, mannitol and sorbitol and mixtures thereof. Preferred organic acids are selected from the group consisting of acetic acid, propionic acid, lactic acid, formic acid, malic acid, citric acid, fumaric acid and higher alkanoic acids and mixtures thereof. Chemically undefined mixtures of compounds may be extracts from microbial, animal or plant material or cells or created from such material or cells, e.g., plant protein preparations, soy meal, corn meal, pea meal, corn gluten, corn steep liquor, cotton meal, peanut meal, potato meal, meat, casein, gelatins, whey, fish meal, yeast protein, yeast extract, tryptone, peptone, bacto-tryptone, bacto-peptone, wastes from the processing of microbial cells, plants, meat or animal bodies, and combinations thereof. Depending on the recombinant bacterial cells to be used, i.e. the platform recombinant bacterial cells whose growth and/or viability is dependent on the presence or certain amount of an inducer molecule in the culture medium or the producing bacterial cells obtained after transformation with an expression plasmid as specified herein whose growth and/or viability in the absence of the inducer molecule in the culture medium depend on proper uptake of the expression plasmid, a culture medium to be used in accordance with the present invention may or may not comprise further ingredients such as an inducer molecule.

A bioreactor as referred to herein is a vessel in which the cultivation of cells in large scale and the large-scale production of a product of interest takes place. After termination of the cultivation in the production bioreactor the fermentation broth is harvested and the product of interest is recovered. The bioreactor may contain inlets and outlets, for example for media, and different sensors, e.g. for measuring pH and temperature during the fermentation process. The fermentation medium in the production bioreactor may be the same as or different from the fermentation medium used in the seed fermenter or the last seed fermenter in a seed train. The production bioreactor may have a volume of 500 L, 1,000 L, 5,000 L, 10,000 L, 20,000 L, 50,000 L or 100,000 L or more.

The cultivation as referred to before may be carried out in a batch mode wherein the cells are cultured in the initially present culture medium without any change in medium composition or the volume of the medium. Thus, in batch mode no substantial or significant amount of fresh liquid culture medium is added to the cell culture and no substantial or significant amount of liquid culture medium is removed from the cell culture during culturing. Alternatively, the culture may be carried out in a fed-batch mode wherein the cells are cultured in the initially present culture medium and a feed solution is added in a periodic or continuous manner without substantial or significant removal of liquid culture medium during culturing. Fed-batch cultures can include various feeding regimens and times, for example, daily feeding, feeding more than once per day, or feeding less than once per day, and so on. Yet, as an alternative, the culture may be carried out in a continuous fermentation mode wherein the cells are cultured in the initially present culture medium and new culture medium is continuously fed to the bioreactor and fermented culture medium is removed from the bioreactor at the same rate so that the volume in the bioreactor is constant. The skilled worker knows that such batch, semi-batch or continuous fermentation processes can be done without nutrition limitations during the fermentation process, however in general such processes are run by limiting at least one nutrition during the fermentation or during a certain period of the fermentation in general such limitations are made by restricting for example the C-, N- and/or P-source or by limiting oxygen.

Thus, in a preferred embodiment of the aforementioned method of the invention, said method further comprises:
(c) obtaining the compound of interest manufactured by the recombinant bacterial cell.

After the aforementioned method of the present invention has been performed, the compound of interest may be obtained by purification from the culture medium used for culture or from the cultivated recombinant bacterial cell(s). Typically, the fermentation broth and the bacterial cells are separated from each other, e.g., by centrifugation, sedimentation or filtration techniques.

If the compound of interest is present in the culture medium, further purification steps may depend on the chemical nature and properties of the compound of interest. Such purification steps may encompass affinity chromatography (e.g., ion exchange-, affinity-, hydrophobic-, chromatofocusing-, and size exclusion-chromatography), extraction, electrophoresis (e.g., preparative isoelectric focusing), filtration, evaporation, spray-drying, precipitation (e.g., using ammonium sulfate) or crystallization or combinations thereof.

If the compound of interest is comprised in the recombinant bacterial, release of the compound of interest from said cells might be needed. Release from the cells can be achieved, for instance, by cell lysis with techniques well known to the skilled artisan, e.g., lysozyme treatment, ultrasonic treatment, French press or combinations thereof. Subsequently, the compound of interest released from the recombinant bacterial cells may be further purified by purification steps as referred to above. Preferably, for the purification of pDNA vectors, several types of chromatographic methods (e.g., hydrophobic interaction, anion exchange, multimodal, affinity) can be applied after primary isolation. Details of said methods are well known in the art (see, e.g., Alves at al. 2021 and references therein).

The method of the present invention may also encompass further steps for formulating the compound of interest in a desired form. This may include the generation of granulates, tablets, powders, solutions, gels, gaseous formulations, any 3D-items, nanoparticles, coatings and the like from the compound of interest.

Depending on the nature of the compound of interest, the formulation of the compound of interest may be carried out under particular standard conditions, such as GMP.

The present invention also relates to the use of the recombinant bacterial cell of the invention for the recombinant manufacture of a compound of interest, in general.

Yet, the present invention provides for a kit for recombinant manufacture of a compound of interest comprising:
(i) a recombinant bacterial cell of the invention, and
(ii) an expression plasmid which comprises at least one plasmid copy of the at least one essential gene, wherein said plasmid copy is under the control of the expression control sequence which is biologically active in said bacterial cell.

The term “kit” as used herein refers to a collection of components required for carrying out the method of the present invention for recombinant manufacture of a compound of interest. The kit shall include a recombinant bacterial cell of the present invention and an expression plasmid which comprises at least one plasmid copy of the at least one essential gene, wherein said plasmid copy is under the control of the expression control sequence which is biologically active in said bacterial cell. Preferably, said expression control sequence which is biologically active is the expression control sequence which is natively associated with the at least one endogenous essential gene. Also preferably, said expression plasmid comprises at least one further nucleic acid of interest which is required for the recombinant manufacture of a compound of interest in said recombinant bacterial cell which is required for the recombinant manufacture of a compound of interest in said recombinant bacterial cell.

Typically, the components of the kit are provided in separate containers or within a single container. The container also typically comprises instructions for carrying out the method of the present invention for recombinant manufacture of a compound of interest. Moreover, the kit may, preferably, comprise further components which are necessary for carrying out the method of the invention such as cultivation media, washing solutions, solvents, and/or reagents or means required for purification of the compound of interest.

The following embodiments are particularly preferred embodiments envisaged in accordance with the present invention. All definitions and explanations of the terms made above apply mutatis mutandis.

Embodiment 1. A recombinant bacterial cell comprising in its genome at least one essential gene the endogenous promoter of which has been replaced by a heterologous expression control sequence being inducible by an inducer molecule, such that the expression of said essential gene in the recombinant bacterial cell is dependent on the presence of said inducer molecule.

Embodiment 2: The recombinant bacterial cell of embodiment 1, wherein said sat least one essential gene (i) is an absolute essential gene which is absolutely required for proper cell growth, proliferation and/or survival even under optimal growth conditions or which is indispensable for DNA replication, RNA synthesis, protein synthesis, central metabolism, cell wall or membrane synthesis or reproduction or (ii) is a combination of essential genes which are in combination absolutely required for proper cell growth, proliferation and/or survival even under optimal growth conditions or which is indispensable for DNA replication, RNA synthesis, protein synthesis, central metabolism, cell wall or membrane synthesis or reproduction.

Embodiment 3. The recombinant bacterial cell of embodiment 1 or 2, wherein said bacterial cell is from a genus selected from the group consisting of Escherichia, Vibrio, Bacillus, Lactobacillus, Acetobacter, Corynebacterium, Brevibacterium, Pseudomonas, Streptomyces, Gluconobacter, Clostridium, Streptococcus, Zooepidemicus, Basfia, Crytococcus, Rhodotorula, Nocardia, Erwinia, Xanthomonas, Leuconostoc and Klebsiella, preferably, is an E. coli cell.

Embodiment 4. The recombinant bacterial cell of any one of embodiments 1 to 3, wherein said at least one essential gene is a gene required for cell growth and/or viability, preferably, selected from the group consisting of: infA, infC/IF-3, dnaJ, dnaK, era, frr, ftsL, ftsN, ftsZ, grpE, mopA, mopB, msbA, nusG, parC, rpsB, secY and trmA.

Embodiment 5. The recombinant bacterial cell of embodiment 4, wherein said at least one essential gene is infA, secY or frr.

Embodiment 6. The recombinant bacterial cell of any one of embodiments 1 to 5, wherein the bacterial genome lacks any gene being capable of functionally complementing the at least one essential gene in the absence of the inducer molecule.

Embodiment 7. The recombinant bacterial cell of any one of embodiments 1 to 6, wherein said bacterial cell comprises an expression plasmid which comprises at least one plasmid copy of the at least one essential gene, wherein said plasmid copy is under the control of the expression control sequence which is biologically active in said bacterial cell.

Embodiment 8. The recombinant bacterial cell of embodiment 7, wherein said expression control sequence which is biologically active is the expression control sequence which is natively associated with the at least one endogenous essential gene.

Embodiment 9. The recombinant bacterial cell of embodiment 7 or 8, wherein said expression plasmid comprises at least one further nucleic acid of interest which is required for the recombinant manufacture of a compound of interest in said recombinant bacterial cell.

Embodiment 10. The recombinant bacterial cell of any one of embodiments 1 to 9, wherein said inducer molecule is selected from the group consisting of arabinose, rhamnose, xylose, sucrose, tetracycline, anhydrotetracycline and IPTG.

Embodiment 11. A method for generating the recombinant bacterial cell of any one of embodiments 1 to 10 comprising the steps of:
(a) exchanging in the genome of a bacterial cell the endogenous promoter of at least one essential gene by a first polynucleotide comprising a selection cassette comprising a promoter that can be active in the bacterial cell operatively linked to
   (i) a first selection gene that confers sensitivity for a first selection agent such that the bacterial cell will not grow and/or will die in the presence of said first selection agent, and
   (ii) a second selection gene that confers resistance for a second selecting agent such that the bacterial cell will be able to grow in the presence of said second selecting agent,
   such that the expression of the at least one essential gene, the first and the second selection gene will become dependent on the said promoter;
(b) selecting a bacterial cell which has integrated into its genome said first polynucleotide using the second selecting agent;
(c) exchanging said first polynucleotide which has been integrated into the genome by a second polynucleotide comprising a heterologous expression control sequence being inducible by an inducer molecule such that the expression of the at least one essential gene becomes dependent on said heterologous expression sequence; and
(d) selecting a bacterial cell which has integrated into its genome said second polynucleotide using the first selection molecule and the inducer molecule.

Embodiment 12. The method of embodiment 11, wherein said first selection gene is selected from the group consisting of: rpsL, sacB, pheS, tetR, galK, thyA, tolC and ccdB.

Embodiment 13. The method of any one of embodiments 11 or 12, wherein said first selection agent is selected from the group consisting of streptomycin, sucrose, chloro-phenylalanine, chlortetracycline and fusaric acid, galactose, trimethoprim and derivatives thereof, and bacteriocins, preferably, colicins, preferably, colicin A, B, E1, Ia, Ib, N, D, M or E3, more preferably colicin E1.

Embodiment 14. The method of any one of embodiments 11 to 13, wherein said second selection gene is selected from the group consisting of: kmR, ampR, zeoR, tetR, cmR, hygR, specR, and BSD.

Embodiment 15. The method of any one of embodiments 11 to 14, wherein said second selection agent is selected from the group consisting of: kanamycin, ampicillin, zeocin, tetracycline, chloramphenicol, hygromycin, spectinomycin, and blasticidin.

Embodiment 16. The method of any one of embodiments 11 to 15, wherein the bacterial cell is cultivated after step (d) in the presence of the inducer molecule.

Embodiment 17. The method of any one of embodiments 11 to 16, wherein said selection cassette is further comprising a restriction enzyme cleavage site at the 3′end of the second selection gene.

Embodiment 18. The method of embodiment 17, wherein said method further comprises after step (c) the step of treating the bacterial cell with the restriction enzyme such that a genome of a bacterial cell still comprising the first polynucleotide comprising the selection cassette will be cleaved between the second selection gene and the at least one essential gene while the genome of a bacterial cell having exchanged the first polynucleotide comprising a selection cassette which has been integrated into the genome by a second polynucleotide comprising a heterologous expression control sequence being inducible by an inducer molecule such that the expression of the at least one essential gene becomes dependent on said heterologous expression sequence remains unaffected by the restriction enzyme.

Embodiment 19. The method of embodiment 17 or 18, wherein said restriction enzyme is selected from the group consisting of: I-SceI, I-CeuI, I-PpoI, PI-SceI, andPI-PspI.

Embodiment 20. A method for generating the recombinant bacterial cell of any one of embodiments 1 to 10 comprising the steps of:
(a) exchanging in the genome of a bacterial cell the endogenous promoter of at least one essential gene by a polynucleotide comprising
   (i) a selection gene that confers resistance for a selection agent, said selection gene being flanked by recognition sites for site specific recombination in the bacterial cell, and
   (ii) a heterologous expression control sequence being inducible by an inducer molecule,
   such that the expression of the at least one essential gene becomes dependent on said heterologous expression control sequence and such that the bacterial cell will grow in the presence of said inducer molecule and in the presence of said selection agent;
(b) selecting a bacterial cell which has integrated into its genome said polynucleotide using the selection agent and the inducer molecule;
(c) removing the selection gene by using a recombinase that is capable of carrying out site specific recombination using the recognition sites flanking the selection gene such that the expression of the at least one essential gene remains dependent on said heterologous expression control sequence; and
(d) selecting a bacterial cell which has integrated into its genome said polynucleotide lacking the selection gene using the inducer molecule.

Embodiment 21. The method of embodiment 20, wherein said selection gene is selected from the group consisting of: ampR, kmR, zeoR, tetR, cmR, hygR, specR, and BSD.

Embodiment 22. The method of embodiment 20 or 21, wherein said selection agent is selected from the group consisting of ampicillin, kanamycin, zeocin, tetracycline, chloramphenicol, hygromycin, spectinomycin, and blasticidin.

Embodiment 23. A method for generating the recombinant bacterial cell of any one of embodiments 1 to 10 comprising the steps of:
(a) exchanging in the genome of a bacterial cell the endogenous promoter of at least one essential gene by a polynucleotide comprising
   (i) a heterologous expression control sequence being inducible by an inducer molecule,
   (ii) a promoter that can be active in the bacterial cell, and
   (iii) a selection gene that confers resistance for a selection agent, wherein a segment of the polynucleotide comprising (ii) the promoter that can be active in the bacterial cell and (iii) the selection gene but not the heterologous expression control sequence is flanked by recognition sites for site specific recombination in the bacterial cell,
   such that the expression of the at least one essential gene will become dependent on the promoter that can be active in the bacterial cell;
(b) selecting a bacterial cell which has integrated into its genome said polynucleotide using the selection agent;
(c) removing the segment of the polynucleotide comprising the promoter that can be active in the bacterial cell and the selection gene by using a recombinase that is capable of carrying out site specific recombination using the recognition sites flanking the segment of the polynucleotide such that the expression of the at least one essential gene becomes dependent on said heterologous expression control sequence; and
(d) selecting a bacterial cell which has integrated into its genome said polynucleotide lacking the segment flanked by the recognition sites using the inducer molecule.

Embodiment 24. The method of embodiment 23, wherein said selection gene is selected from the group consisting of: kmR, ampR, zeoR, tetR, cmR, hygR, specR, and BSD.

Embodiment 25. The method of embodiment 23 or 24, wherein said selection agent is selected from the group consisting of: kanamycin, ampicillin, zeocin, tetracycline, chloramphenicol, hygromycin, spectinomycin, and blasticidin.

Embodiment 26. The method of any one of embodiments 20 to 25, wherein said recognition sites for site specific recombination in the bacterial cell are FRT or loxP sequences.

Embodiment 27. The method of any one of embodiments 20 to 26, wherein said recombinase is a FLP recombinase or a Cre recombinase.

Embodiment 28. The method of any one of embodiments 11 to 27, wherein said promoter that can be active in the bacterial cell is a promoter constitutively active in the bacterial cell or an inducible promoter.

Embodiment 29. The method of embodiment 28, wherein said promoter constitutively active in the bacterial cell is selected from the group consisting of: rpsL promoter, infA promoter, infAp2 promoter, em7 promoter, and cat promoter.

Embodiment 30. The method of embodiment 28, wherein said inducible promoter is selected from the group consisting of a heat-inducible promoter, tetracycline-inducible promoter, a lacI/IPTG promoter, an ethanol-inducible promoter (AlcA/AlcR), a steroid-inducible promoter (LexA/XVE), and a vanillate-inducible promoter.

Embodiment 31. The recombinant bacterial cell of any one of embodiments 1 to 10 or the method of any one of embodiments 11 to 30 wherein said heterologous expression control sequence being inducible by an inducer molecule is selected from the group consisting of: AraC/PBAD promoter, RhaR-RhaS/rhaBAD promoter, XylS/Pm promoter, NitR/PnitA promoter, ChnR/Pb inducer/promoter, a tetracycline-inducible promoter (tetON/OFF), a lacI/IPTG promoter, an ethanol-inducible promoter (AlcA/AlcR), a steroid-inducible promoter (LexA/XVE), and a vanillate-inducible promoter.

Embodiment 32. The method of any one of embodiments 11 to 31, wherein said inducer molecule is selected from the group consisting of: arabinose, rhamnose, xylose, sucrose, tetracycline, anhydrotetracycline and IPTG.

Embodiment 33. A method for recombinant manufacture of a compound of interest comprising the steps of:
(a) introducing into a recombinant bacterial cell of any one of embodiments 1 to 6 an expression plasmid which comprises at least one plasmid copy of the at least one essential gene, wherein said plasmid copy is under the control of the expression control sequence which is biologically active in said bacterial cell; and
(b) cultivating the recombinant bacterial cell obtained in step a) in the absence of the inducer molecule.

Embodiment 34. The method of embodiment 33, wherein said expression plasmid comprises at least one further nucleic acid of interest which is required for the recombinant manufacture of a compound of interest in said recombinant bacterial cell.

Embodiment 35. The method of embodiment 33 or 34, wherein said method further comprises: (c) obtaining the compound of interest manufactured by the recombinant bacterial cell.

Embodiment 36. The method of any one of embodiments 33 to 35, wherein said expression control sequence which is biologically active is the expression control sequence which is natively associated with the at least one endogenous essential gene.

Embodiment 37. Use of the recombinant bacterial cell of any one of embodiments 7 to 10 for the recombinant manufacture of a compound of interest.

Embodiment 38. A kit for recombinant manufacture of a compound of interest comprising:
(i) a recombinant bacterial cell of any one of embodiments 1 to 6, and
(ii) an expression plasmid which comprises at least one plasmid copy of the at least one essential gene, wherein said plasmid copy is under the control of the expression control sequence which is biologically active in said bacterial cell.

Embodiment 39. The kit of embodiment 38, wherein said expression control sequence which is biologically active is the expression control sequence which is natively associated with the at least one endogenous essential gene.

Embodiment 40. The kit of embodiment 38 or 39, wherein said expression plasmid comprises at least one further nucleic acid of interest which is required for the recombinant manufacture of a compound of interest in said recombinant bacterial cell which is required for the recombinant manufacture of a compound of interest in said recombinant bacterial cell.

All references cited in this specification are herewith incorporated by reference with respect to their entire disclosure content and the disclosure content specifically mentioned in this specification.

### FIGURES

FIG. 1: Schematic overview of an approach for generating a producing strain using two selection genes and Red/ET recombination. A) using Red/ET recombination, a first expression cassette comprising in 5′ to 3′order an rpsL promoter, rpsL gene, kmR gene and I-SceI restriction site is recombined with the promoter region of the endogenous infA gene. B) an intermediated strain is obtained by selecting for kanamycin resistance, C) using Red/ET recombination, the first expression cassette is replaced by a second expression cassette comprising in 5′ to 3′order araC and the pBAD promoter, selection for proper recombinant cells is done by selecting for bacterial cells resistant to streptomycin (undesired intermediate strain is sensitive to streptomycin), survival/growth in the presence of arabinose and survival/growth in the presence of I-SceI. The platform strain is propagated in the presence of arabinose.

FIG. 2: Schematic overview of a first approach for generating a producing strain using Red/ET recombination and FLP recombination and one selection gene. A) using Red/ET recombination, an expression cassette comprising in 5′ to 3′order FRT site, ampR gene, FRT site, araC and pBAD promoter is recombined with the promoter region of the endogenous infA gene. B) an intermediated strain is obtained by selecting for ampicillin resistance, said strain grows only in the presence of arabinose C) using FLP recombination, the ampR gene is removed. The platform strain is propagated in the presence of arabinose.

FIG. 3: Schematic overview of a second approach for generating a producing strain using Red/ET recombination and FLP recombination and one selection gene. A) using Red/ET recombination, an expression cassette comprising in 5′ to 3′order araC, pBAD promoter, FRT site, rpsL promoter, kmR gene, and FRT site is recombined with the promoter region of the endogenous infA gene. B) an intermediated strain is obtained by selecting for kanamycin resistance, C) using FLP recombination, the kmR gene and rpsL promoter are removed, said strain grows only in the presence of arabinose. The platform strain is propagated in the presence of arabinose.

FIG. 4: Growth control of the different strains on LB agar plates. A) Position of the strains analysed on the plate. B) Growth control on LB agar without addition of arabinose or antibiotics (left plates), on LB agar conditioned with 0.4% L-arabinose (middle plates) and on LB agar conditioned with 15 μg/ml chloramphenicol (right plates).

FIG. 5: Restriction map of plasmid “pQE-T7 TNFα cmR-infA” (SEQ ID NO: 16).

FIG. 6: Restriction map of plasmid “pQE-T7 TNFα infA” (SEQ ID NO: 17).

FIG. 7: Assessing plasmid maintenance of “pQE-T7 TNFα infA-cmR” by the platform strain “HS996 araC-araBp-infA”. Plasmid DNA was isolated from single colonies after transformation of the plasmid into the platform strain. Antibiotics were neither used to select for cells which obtained the plasmid during electroporation nor to maintain the plasmid in overnight cultures. Plasmid DNA analysed via restriction digest with NotI. All six clones showed the expected banding pattern (747 bp and 5289 bp).

FIG. 8: Restriction digestion of four clones obtained after transformation of the platform strain with plasmid “pQE-TNFα infA”. The plasmid DNA was digested with MluI+PacI. The expected restriction fragments of 2078 bp and 3211 bp were obtained for all four clones analysed.

FIG. 9: Growth control of different platform strains on LB agar plates (left plates) and on LB agar plates conditioned with 0.4% L-arabinose (right plates). On the left half of each plate the platform strain was streaked out. On the right half of each plate the corresponding strain harbors the plasmid “pQE-T7 TNFα infA”. A) displays the growth of platform strain E. coli “BL21(DE3) araC-araBp-FRT-infA”; B) the growth of E. coli “TG10 araC-araBp-FRT-infA”; C) the growth of E. coli “HS996 araC-araBp-FRT-infA”

FIG. 10: Growth control of the platform strain W3110 araC-araBp-FRT-infA on LB agar plates. The possibility to grow on LB agar was determined for the strain itself and after transformation of different plasmids with different copy numbers. A) Position of the strains analysed on the plate. B) Growth control on LB agar without addition of arabinose (left plate) and on LB agar conditioned with 0.4% L-arabinose (right plate).

FIG. 11: Growth control of the platform strain HS996 pir+ araC-araBp-FRT-infA on LB agar. The possibility to grow on LB agar was determined for the strain itself and after transformation of different plasmids with different copy number and different origins of replication (GenH015−plasmid1=R6K origin, GenH015−plasmid2=p15A origin and GenH015−plasmid3=ColE1 origin). A) Position of the strains analysed on the plate. B) Growth control on LB agar without addition of arabinose.

FIG. 12: Restriction maps of the different reporter plasmids. A) Plasmids based on a R6K origin of replication. On the left side “GenH015-plasmid1-cmR” (SEQ ID NO: 23). On the right side “GenH015-plasmid1” (SEQ ID NO: 26). B) Plasmids based on a p15A origin of replication. On the left side “GenH015-plasmid2-cmR” (SEQ ID NO: 24). On the right side “GenH015-plasmid2” (SEQ ID NO: 27). C) Plasmids based on a ColE1 origin of replication. On the left side “GenH015-plasmid3-cmR” (SEQ ID NO: 25). On the right side “GenH015-plasmid3” (SEQ ID NO: 28).

FIG. 13: Schematic overview of the approach for generating a producing strain using Red/ET recombination and FLP recombination based on the frr gene. A) Using Red/ET recombination, an expression cassette comprising in 5′ to 3′order araC, pBAD promoter, FRT site, rpsL promoter, zeoR gene, and FRT site is recombined with the promoter region of the endogenous frr gene. B) An intermediated strain is obtained by selecting for zeocin resistance, C) using FLP recombination, the zeoR gene and rpsL promoter are removed, said strain grows only in the presence of arabinose. The platform strain is propagated in the presence of arabinose

FIG. 14: Growth control of platform strain “HS996 araC-araBp-FRT-frr” on LB agar (left plate) and on LB agar conditioned with 0.4% L-arabinose (right plate). On the left half of each plate the platform strain was streaked out. On the right half of each plate the corresponding strain harbors the plasmid “pQE-T7 TNFα frr”.

FIG. 15: Restriction map of plasmid “pQE-T7 TNFα zeoR-frr” (SEQ ID NO: 35).

FIG. 16: Restriction map of plasmid “pQE-T7 TNFα frr” (SEQ ID NO: 36).

FIG. 17: Growth control of clones propagated for >120 generations without selection. Three different combinations of platform strain and plasmid were analysed: “HS996 pir+ araC-araBp-FRT-infA”/“GenH015-plasmid1” (left plate), “T7E2 araC-araBp-FRT-infA”/“GenH015-plasmid2” (right plate) and “W3110 araC-araBp-FRT-infA”/“GenH015-plasmid3” (middle plate). A small aliquot from the last overnight culture was streaked out onto an LB agar plate and incubated at 37° C. overnight to obtain single colonies. All colonies obtained display the expected violet color indicating the presence of the corresponding plasmid. The color of the colonies obtained from the combination “T7E2 araC-araBp-FRT-infA”/“GenH015-plasmid2” (right plate) is less intense due to the low copy number of the p15A-based plasmid.

FIG. 18: Growth control of clones (platform strain harbouring plasmid “pQE-T7 TNFα zeoR-frr”) propagated for >120 generations without selection for cm-resistance. 24 clones were streaked out on an LB agar plate (left) and on an LB agar plate conditioned with 25 g/ml zeocin to confirm the presence of plasmid “pQE-T7 TNFα zeoR-frr” after seven days of passages without the addition of antibiotics.

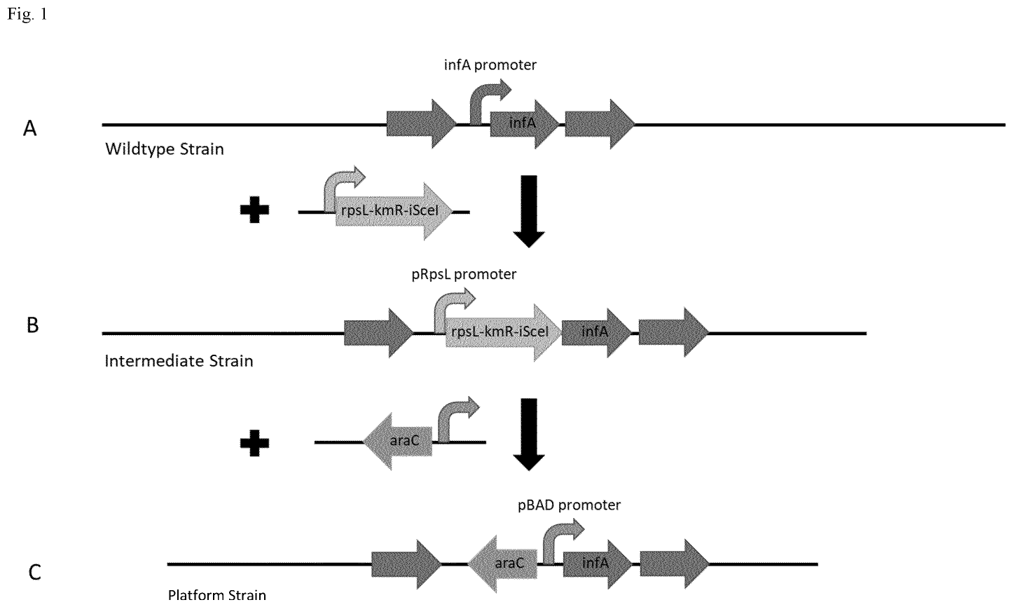

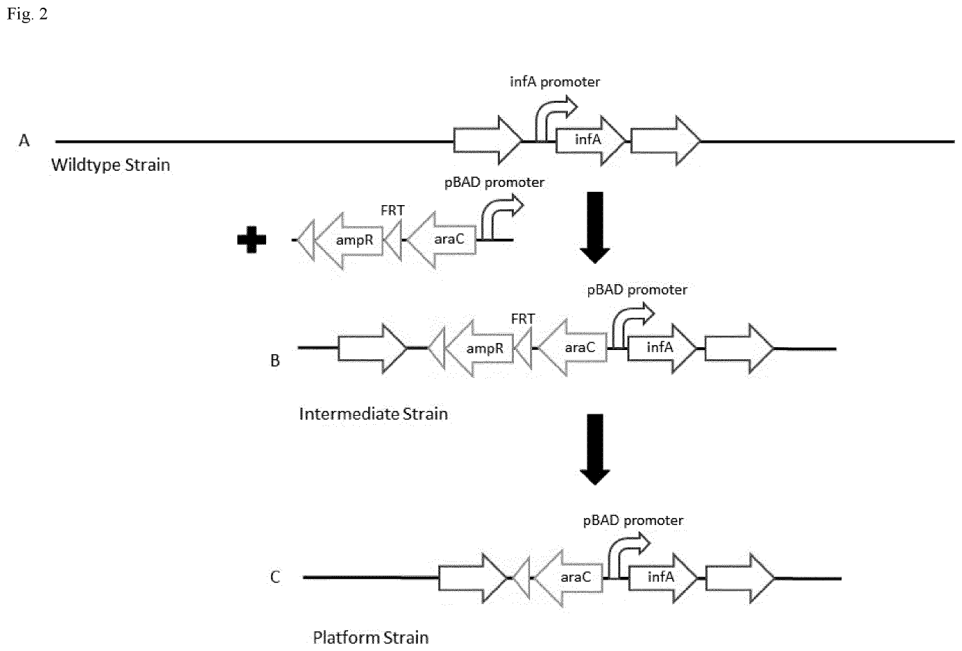

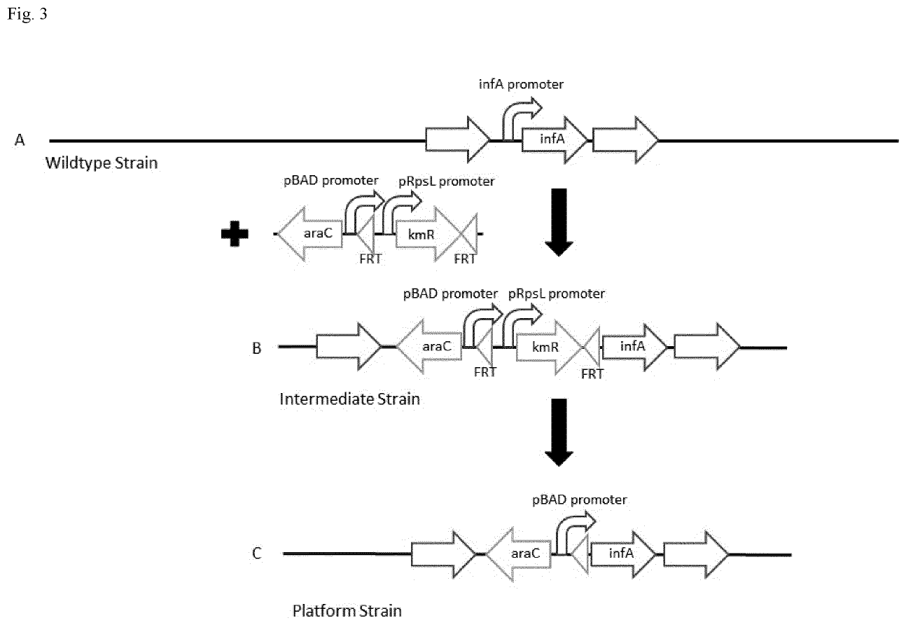

### EXAMPLES

The following Examples shall merely illustrate the invention. They shall not be construed, whatsoever, to limit the scope thereof.

The SEQ ID NOs referred to in the following show:
SEQ ID NO: 1: “rpsL-zeoR” cassette: 219 bp
SEQ ID NO: 2: PCR Primer “CS-primer up”: 86 bp
SEQ ID NO: 3: PCR Primer “CS-primer down”: 77 bp
SEQ ID NO: 4: “araC-araBp-infA” knock-in cassette: 1442 bp
SEQ ID NO: 5: PCR Primer “CS2-primer up”: 70 bp
SEQ ID NO: 6: PCR Primer “CS2-primer down”: 17 bp
SEQ ID NO: 7: genomic infA locus after modification (aat-infA segment): 2064 bp
SEQ ID NO: 8: “FRT-ampR-FRT-araC-araBp” knock-in cassette: 2682 bp
SEQ ID NO: 9: PCR Primer “FRT-primer up”: 70 bp
SEQ ID NO: 10: PCR Primer “FRT-primer down”: 22 bp
SEQ ID NO: 11: genomic infA locus after modification (aat-infA segment): 2184 bp
SEQ ID NO: 12: “araC-araBp-FRT-kmR-FRT” knock-in cassette: 2647 bp
SEQ ID NO: 13: PCR Primer “FRT2-primer up”: 22 bp
SEQ ID NO: 14: genomic infA locus after modification (aat-infA segment): 2114 bp
SEQ ID NO: 15: Escherichia coli infA gene: 219 bp
SEQ ID NO: 16: Escherichia coli secY gene: 1332 bp
SEQ ID NO: 17: Plasmid “pQE-T7 TNF alpha”: 5746 bp
SEQ ID NO: 18: PCR Primer “infA-primer up”: 68 bp
SEQ ID NO: 19: PCR Primer “infA-primer down”: 77 bp
SEQ ID NO: 20: “infA-cmR” expression cassette: 1327 bp
SEQ ID NO: 21: Plasmid “pQE-T7 TNFα cmR-infA”: 6036 bp
SEQ ID NO: 22: Plasmid “pQE-T7 TNFα infA”: 5289 bp
SEQ ID NO: 23: Plasmid “GenH015-plasmid1-cmR”: 3184 bp
SEQ ID NO: 24: Plasmid “GenH015-plasmid2-cmR”: 3417 bp
SEQ ID NO: 25: Plasmid “GenH015-plasmid3-cmR”: 3596 bp
SEQ ID NO: 26: Plasmid “GenH015-plasmid1”: 2437 bp
SEQ ID NO: 27: Plasmid “GenH015-plasmid2”: 2670 bp
SEQ ID NO: 28: Plasmid “GenH015-plasmid3”: 2849 bp
SEQ ID NO: 29: araC-araBp-FRT-zeoR-FRT” knock-in cassette for frr locus: 2423 bp
SEQ ID NO: 30: PCR Primer “FRT-frr-primer up”: 19 bp
SEQ ID NO: 31: PCR Primer “FRT-frr-primer down”: 23 bp
SEQ ID NO: 32: genomic frr locus after modification (pyrH-araC-araBp-FRT-zeoR-FRT-frr segment): 3432 bp
SEQ ID NO: 33: genomic frr locus after FLP recombination (pyrH-araC-araBp-FRT-frr segment): 2888 bp
SEQ ID NO: 34: “zeoR-frr” expression cassette: 1484 bp
SEQ ID NO: 35: Plasmid “pQE-T7 TNFα zeoR-frr”: 6176 bp
SEQ ID NO: 36: Plasmid “pQE-T7 TNFα frr”: 5604 bp
SEQ ID NO: 37: Escherichia coli frr gene: 558 bp

### Example 1: Preparation of a Platform Strain with an Arabinose-Inducible infA Gene

a) Preparation of a Platform Strain by Direct Replacement of the Endogenous infA Promoter by an Arabinose-Inducible Promoter Using the rpsL-Zeo Counter-Selection System

A rpsL-based counter-selection system was applied to replace the endogenous infA promoter by an arabinose-inducible promoter. This selection and counter-selection system was developed by Gene Bridges (“Counter-Selection BAC Modification Kit” Gene Bridges, Cat. No. K002). It takes advantage of the fact that the S12 ribosomal protein is the target of the antibiotic streptomycin. Mutations in the rpsL gene encoding this protein are responsible for resistance to high concentrations of streptomycin (Chumpolkulwong, N. et al. 2004). Resistance is recessive in a merodiploid strain. When both wild-type and mutant alleles of rpsL are expressed in the same strain, the strain is sensitive to streptomycin. Most of the commonly used E. coli strains (e.g., DH10B, HS996, TOP10) carry a mutation in the rpsL gene resulting in streptomycin resistance.

Other strains can easily be converted into a streptomycin resistance phenotype by introducing the necessary point mutation (A/G transition at position 42; K42R) in the rpsL gene by Red/ET recombination (Zhang, Y. et al. 2000; Murphy, K. C., Campellone, K. G. and Poteete, A. R. 2000).

aa) Replacement of the Endogenous infA Promoter by a rpsL-Zeo Counter-Selection Cassette

A DNA fragment of 1026 bp in size consisting of a rpsL gene and its promoter, linked to a zeocin resistance marker gene and flanked by 50 bp long homology arms to the infA genomic locus (SEQ ID NO: 1) was amplified using the primers CS-primer up (SEQ ID NO: 2) and CS-primer down (SEQ ID NO: 3).

Red/ET recombination (Zhang, Y. et al. 2000; Murphy, K. C., Campellone, K. G. and Poteete, A. R. 2000) was used to insert the obtained PCR product upstream of the infA start codon of E. coli strain HS996, replacing the endogenous promoter. The recombineering reaction was performed as described in the manual of the “Quick & Easy E. coli Gene Deletion Kit” (Gene Bridges, Cat. No. K006). Selection for cells with chromosomal integration of the cassette was performed on LB agar plates containing 25 μg/ml zeocin. The correctly performed knock-in was confirmed by PCR across the modified infA locus and subsequent sequencing of the amplicon. In addition, the cells were analyzed for their ability to grow on LB agar plates containing 200 μg/ml streptomycin. Only colonies that were sensitive for streptomycin were chosen for the subsequent counter-selection step.

ab) Replacement of the rpsL-Zeo Counter-Selection Cassette by an araC-araBp Cassette to Obtain an Arabinose-Inducible infA Gene

A DNA fragment of 1442 bp in size consisting of the araC gene together with the araB promoter, and 50 bp/119 bp long homology arms to the infA genomic locus at the end was amplified using the primers CS2-primer up (SEQ ID NO: 5) and CS2-primer down (SEQ ID NO: 6). The obtained PCR product (SEQ ID NO: 4) was inserted between the aat gene and the infA gene by Red/ET recombination. The sequence of the genomic locus after the insertion of the arabinose-inducible promoter is given in SEQ ID NO:7.

The Red/ET recombination step was performed following the instructions of the manufacturer with the exception that 0.4% L-arabinose was added to the medium directly after transformation of the cells to induce expression of infA by the araB promoter. Cells were grown for two hours at 37° C. in 1 mL LB medium supplemented with 0.4% L-arabinose but without the addition of antibiotics.

Selection for cells with the chromosomal integration of the cassette was performed on LB agar plates containing 200 μg/ml streptomycin and 0.4% L-arabinose. The plates were incubated overnight at 37° C. The next day, single colonies were patched on two different LB agar plates, where one plate was condition with 0.4% L-arabinose and 25 μg/ml zeocin, and the other plate with only 0.4% L-arabinose. Colonies sensitive for zeocin were further analysed. The correctly performed knock-in of the araC-araBp cassette was confirmed by PCR and subsequent sequencing of the amplicon.

### ac) Growth Control of the Platform Strain

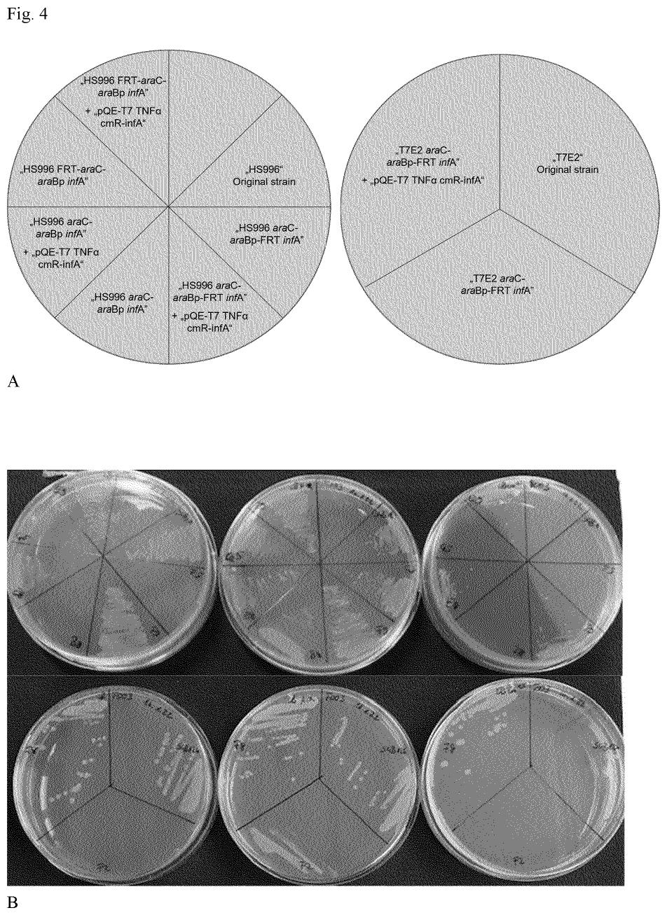

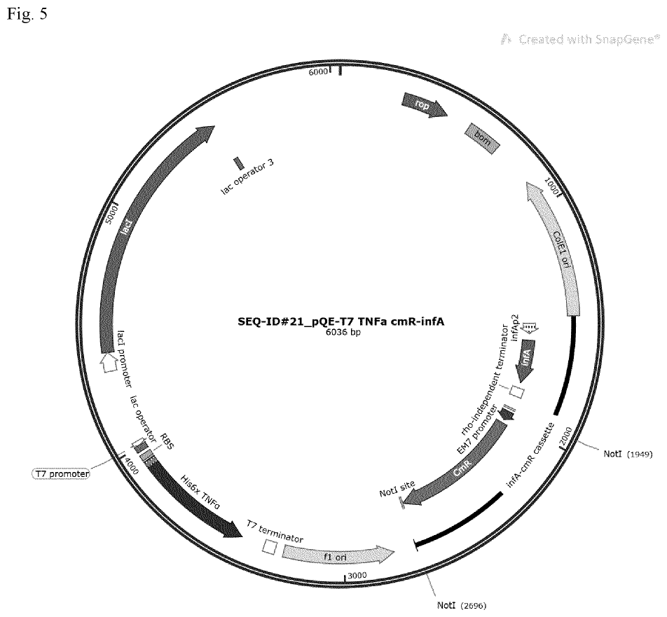

Cells were streaked out on LB agar plates with and without the addition of 0.4% L-arabinose to confirm that the obtained platform strain grows only in the presence of L-arabinose (FIG. 4). E. coli strain “HS996 araC-araBp-infA” is based on the E. coli K12-strain HS996. The platform strain does not carry any antibiotic resistance marker genes in the genome. The resistance for streptomycin used in the protocol above does not stem from an antibiotic resistance gene but rather is the result of the K43T point mutation in the endogenous ribosomal S12.

b) Preparation of a Platform Strain with the Assistance of a Selection Marker Flanked by Recognition Sites for a Site-Specific Recombinase.

Replacement of the endogenous infA promoter by an arabinose-inducible promoter can be performed using a DNA fragment consisting of araC-araBp linked to a selection marker flanked with FRT-sites (FLP Recombination Target sites). FRT-sites are 34 bp DNA sequences comprising two 13 bp palindromes separated by an asymmetric 8 bp core. A sites-specific recombinase (“FLP”) originally isolated from Saccharomyces cerevisiae mediates recombination between two FRT sites (Kilby et al. 1993), such that the intervening DNA fragment (e.g., a selection marker) will be deleted, leaving a single 34 bp FRT site behind. To replace the endogenous infA promoter by an arabinose-inducible promoter, an FRT-flanked selection marker was linked either upstream or downstream to the arabinose-inducible promoter sequence, and the DNA fragment was inserted in the genome via Red/ET recombination at the infA promoter locus. After the replacement of the endogenous infA promoter, an FLP-recombination step was performed, removing the antibiotic resistance marker gene. One FRT site remained either upstream of the inducible promoter in the genome or downstream between the promoter and the start codon of infA.

ba) Replacement of the Endogenous infA Promoter by an FRT-ampR-FRT-araC-araBp Cassette

A DNA fragment of 2682 bp in size consisting of an FRT-flanked ampR resistance marker gene linked to araC-araBp flanked by homology arms to the infA genomic locus (SEQ ID NO: 8) was amplified using the primers FRT-primer up (SEQ ID NO: 9) and FRT-primer down (SEQ ID NO: 10).

Red/ET recombination (Zhang, Y. et al. 2000; Murphy, K. C., Campellone, K. G. and Poteete, A. R. 2000) was used to insert the obtained PCR product upstream of the infA start codon of E. coli strain HS996, replacing the endogenous promoter. The recombineering reaction was performed as described in the manual of the “Quick & Easy E. coli Gene Deletion Kit” (Gene Bridges, Cat. No. K006) with the exception that 0.4% L-arabinose was added to the medium directly after transformation of the cells with the DNA fragment (SEQ ID NO: 8) to induce expression of infA. Cells were grown for two hours at 37° C. in 1 mL LB medium supplemented with 0.4% L-arabinose. Selection for cells with chromosomal integration of the cassette was performed on LB agar plates containing 0.4% L-arabinose and 50 μg/ml ampicillin. The correctly performed knock-in was confirmed by PCR across the modified infA locus and subsequent sequencing of the amplicon.

The ampicillin resistance marker was subsequently removed by FLP-recombination as described in section bc. The sequence of the genomic locus after the insertion of the arabinose-inducible promoter and the FLP-recombination is given in SEQ ID NO:11.

bb) Replacement of the Endogenous infA Promoter by an araC-araBp-FRT-kmR-FRT Cassette

A DNA fragment of 2647 bp in size consisting of araC-araBp linked to an FRT-flanked kmR resistance marker gene and flanked by homology arms to the infA genomic locus (SEQ ID NO: 12) was amplified using the primers FRT2-primer up (SEQ ID NO: 13) and FRT-primer down (SEQ ID NO: 10).

Red/ET recombination (Zhang, Y. et al. 2000; Murphy, K. C., Campellone, K. G. and Poteete, A. R. 2000) was used to insert the obtained PCR product upstream of the infA start codon of E. coli strain T7E2, replacing the endogenous promoter. The recombineering reaction was performed as described in the manual of the “Quick & Easy E. coli Gene Deletion Kit” (Gene Bridges, Cat. No. K006) with the exception that 0.4% L-arabinose was added to the medium directly after transformation of the cells with the DNA fragment (SEQ ID NO: 12) to induce expression of infA. Cells were grown for two hours at 37° C. in 1 mL LB medium supplemented with 0.4% L-arabinose. Selection for cells with chromosomal integration of the cassette was performed on LB agar plates containing 0.4% L-arabinose and 15 μg/ml kanamycin. The correctly performed knock-in was confirmed by PCR across the modified infA locus and subsequent sequencing of the amplicon.

The same protocol was applied to prepare further platform strains based on E. coli BL21(DE3), W3110, TG10 and HS996 pir+.

The kanamycin resistance marker was subsequently removed by FLP-recombination as described in section bc. The sequence of the genomic locus after the insertion of the arabinose-inducible promoter and the FLP-recombination is given in SEQ ID NO:14.

### bc) Removal of the Resistance Marker Gene by FLP-Recombination

The FLP recombination step was performed as described in the manual of the “Quick & Easy E. coli Gene Deletion Kit” (Gene Bridges, Cat. No. K006) with the exception that 0.4% L-arabinose was added to the medium during all steps. The FLP-expression plasmid “708-FLPe cmR” (Gene Bridges, Cat. No. A105) was chosen for the removal of the resistance marker.

Cells were transformed with the FLP-expression plasmid and grown for two hours at 30° C. in 1 mL LB medium supplemented with 0.4% L-arabinose but without the addition of antibiotics. Selection for cells harboring the FLP-expression plasmid was performed on LB agar plates containing 15 μg/ml chloramphenicol and 0.4% L-arabinose. The plates were incubated overnight at 30° C. The next day, 1 mL LB medium supplemented with 0.4% L-arabinose was inoculated with a single colony and incubated for 3 hours at 30° C. Subsequently the incubation temperature was increased to 37° C. and the incubation was continued for another 3 hours. An aliquot of the culture was streaked out on LB agar plates conditioned with 0.4% L-arabinose and the plates were incubated overnight at 37° C. The obtained colonies were analyzed for FLP-mediated marker removal by assessing for sensitivity to the corresponding antibiotic. The correctly performed removal of the antibiotic resistance marker gene was confirmed by PCR and subsequent sequencing of the amplicon.

### bd) Growth Control of the Platform Strain

The growth controls of the platform strains were performed as described in section ac. Cells were streaked out on LB agar plates with and without the addition of 0.4% L-arabinose to confirm that the obtained platform strain grows only in the presence of L-arabinose (FIG. 4). Two platform strains based on infA were prepared as described above using two E. coli strain backgrounds. E. coli strain “T7E2 araC-araBp-FRT-infA” is based on the strain T7E2, which is a derivative of BL21(DE3) lacking approximately 89% of the DE3 prophage. E. coli strain “HS996 FRT-araC-araBp-infA” is based on the E. coli K12-strain HS996. The platform strains do not carry any antibiotic resistance marker genes in the genome.

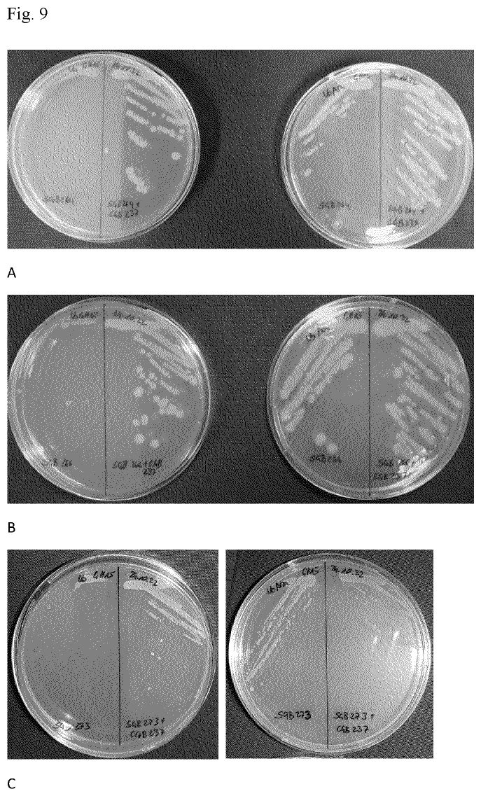

Four additional platform strains based on infA were prepared as described above using the following E. coli strain backgrounds: BL21(DE3), W3110, TG10 and HS996 pir+. All of them grow only in the presence of L-arabinose (FIG. 9-11).

### Example 2: Preparation of Expression Plasmids

a) Preparation of an Expression Plasmid “pQE-T7 TNFα cmR-infA”

Plasmid “pQE-T7 TNFα” (SEQ ID NO: 17) was chosen to confirm that a plasmid encoding the infA gene will be maintained in the platform strains without antibiotic selection. A DNA fragment consisting of a chloramphenicol resistance marker gene flanked by NotI sites was linked to a genomic infA fragment and subsequently amplified using primers infA-primer up (SEQ ID NO: 18) and infA-primer down (SEQ ID NO: 19). The obtained PCR product (SEQ ID NO:20) was recombined with plasmid “pQE-T7 TNFα” using the recombineering proficient E. coli strain GB08-red (Fu et al. 2010). The plasmid modification was performed as described in the publication of Fu and co-workers. The fifty base pair long homology arms for the recombination with plasmid “pQE-T7 TNFα” were chosen to replace the kmR marker in the original plasmid by the “cmR-infA” cassette. The obtained plasmid “pQE-T7 TNFα cmR-infA” (FIG. 5, SEQ ID NO: 21) was used as a first test plasmid for the subsequent plasmid maintenance experiments.

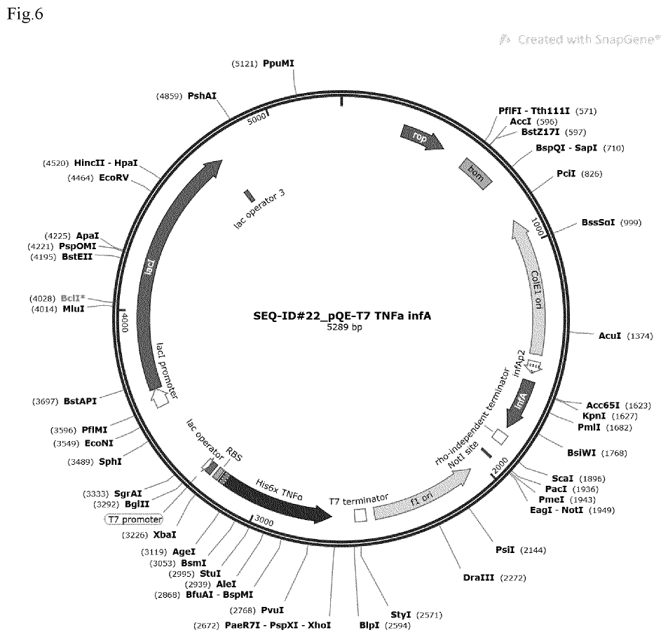

An aliquot of 100 ng plasmid DNA from “pQE-T7 TNFα cmR-infA” was digested with the restriction enzyme NotI and subsequently religated to obtain a test plasmid without an antibiotic resistance marker gene. This second test plasmid was named “pQE-T7 TNFα infA” (FIG. 6, SEQ ID NO: 22).

b) Preparation of Reporter Plasmids with Different Origins of Replication

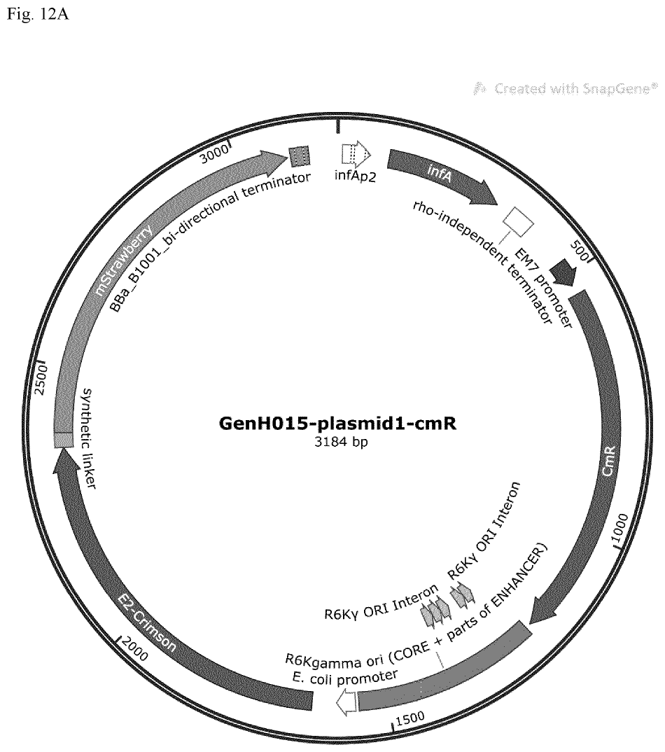

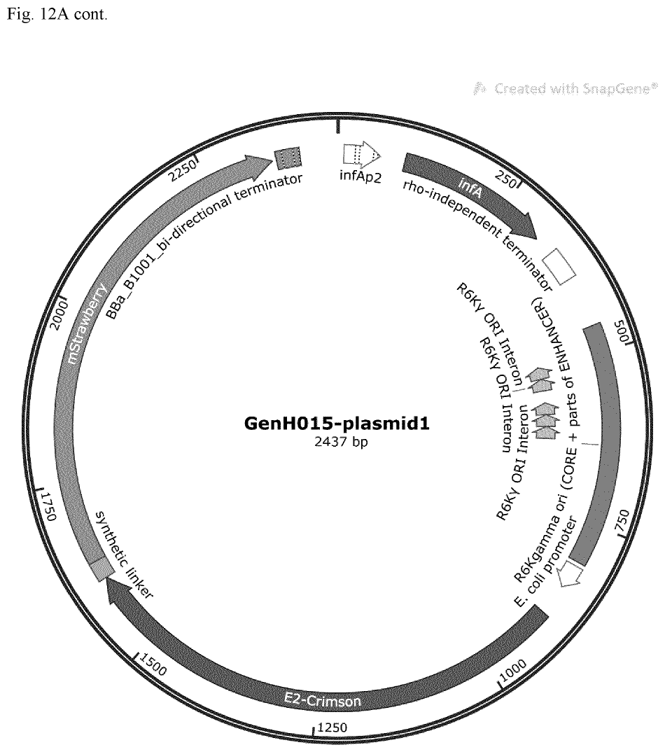

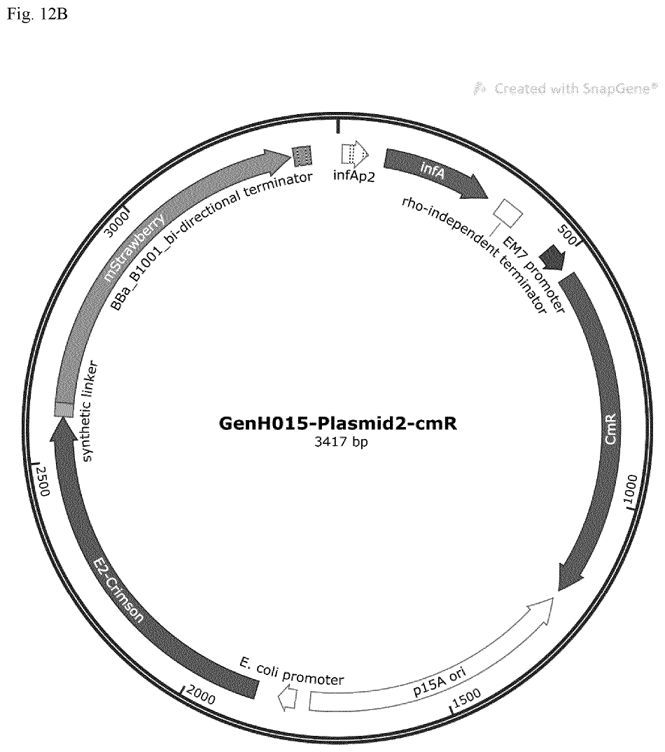

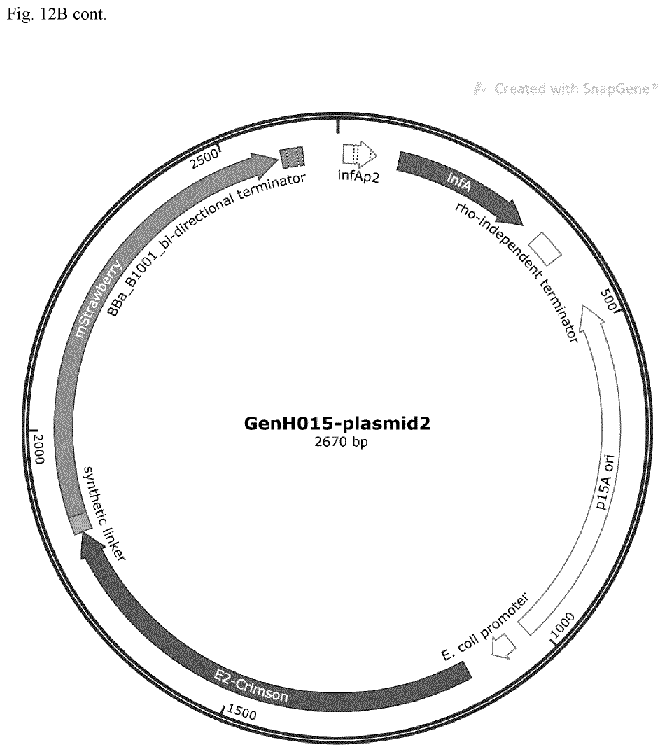

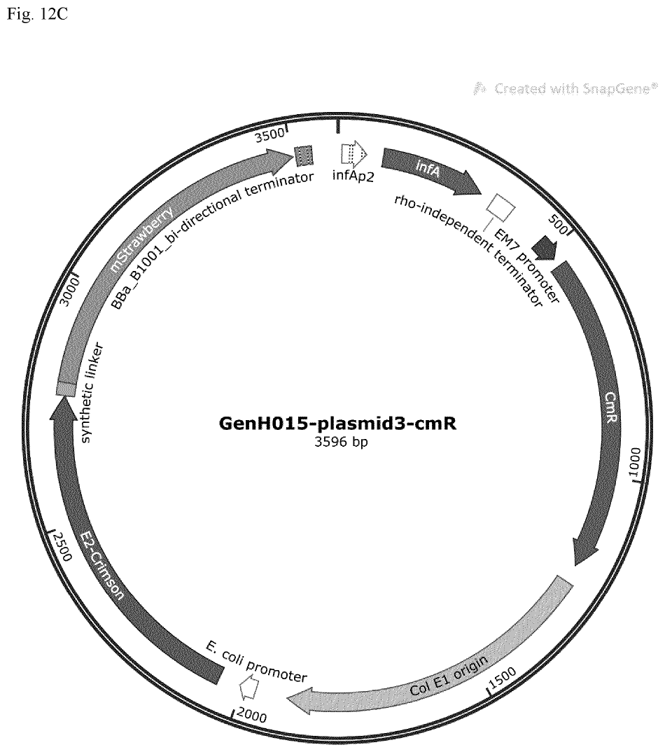

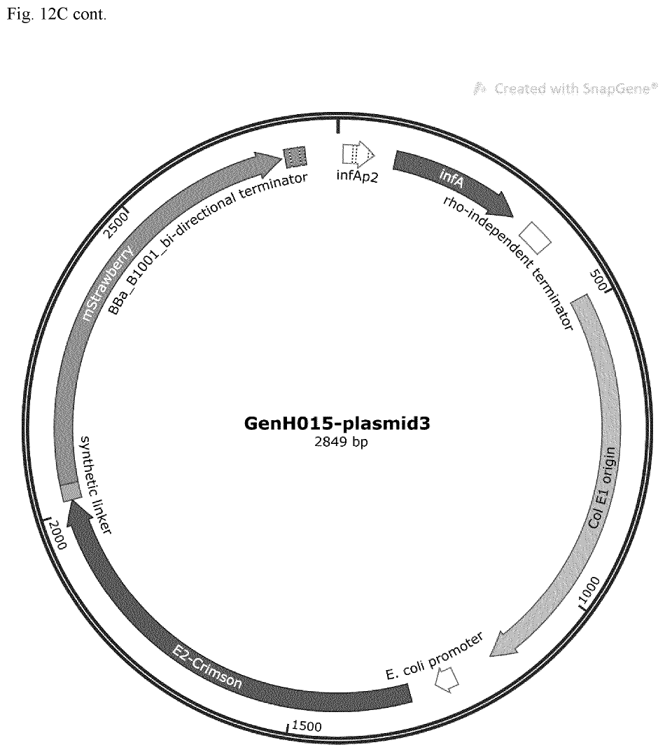

Three additional plasmids “GenH015-plasmid1-cmR”, “GenH015-plasmid2-cmR” and “GenH015-plasmid3-cmR” (FIG. 12, SEQ ID NO: 23-25) were assembled consisting of a chloramphenicol resistance marker gene flanked by NotI sites, the genomic infA fragment already used for “pQE-T7 TNFα infA”, a fluorescent marker cassette and either an R6K, p15A or ColE1 origin or replication. A violet coloration of a clone indicates the presence of the reporter plasmid while E. coli cells without the plasmid will stay white.

An aliquot of 100 ng DNA from each plasmid was digested with NotI and subsequently religated to obtain the corresponding reporter plasmid without an antibiotic resistance marker gene. The three obtained plasmids were named “GenH015-plasmid1”, “GenH015-plasmid2” and “GenH015-plasmid3” (FIG. 12, SEQ ID NO: 26-28).

### Example 3: Confirmation of Plasmid Maintenance Based on “pQE-T7 TNFα infA”

Electrocompetent cells were prepared from the platform strain “T7E2 araC-araBp-FRT-infA” as described in the manual of the “Quick & Easy E. coli Gene Deletion Kit” (Gene Bridges, Cat. No. K006). Approximately 10 ng of the plasmid “pQE-T7 TNFα infA-cmR” was electroporated into the cells of the platform strains. The cells were resuspended after electroporation in 1 ml LB medium (without antibiotics or L-arabinose) and incubated for two hours at 37° C. with shaking at 800 rpm. After this recovery phase the cells were streaked out on LB agar plates (without antibiotics or L-arabinose) and incubated overnight at 37° C. More than one hundred colonies were visible on the plates the next day.

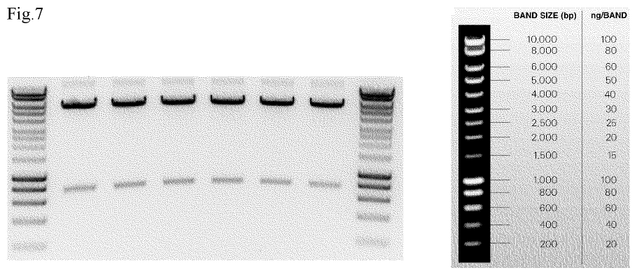

Six colonies were inoculated in LB medium without the addition of antibiotics and grown overnight at 37° C. with 800 rpm. Plasmid DNA was isolated from these clones and analysed with restriction enzymes to confirm the presence of the plasmid in the cells. All analysed colonies display the expected restriction pattern (FIG. 7).

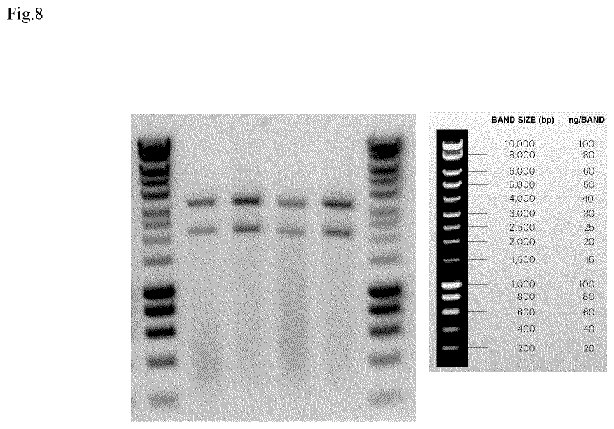

The experiment was repeated with plasmid “pQE-T7 TNFα infA”. More than 50 colonies were obtained. Four colonies were inoculated in LB medium without the addition of antibiotics and grown overnight at 37° C. with 800 rpm. Plasmid DNA was isolated from these clones and analysed with restriction enzymes to confirm the presence of the plasmid in the cells. All analysed colonies display the expected restriction pattern (FIG. 8).

No antibiotic selection was used to insert or keep the plasmid in the cells. Cells are only viable in the presence of a plasmid that carries a copy of the infA gene driven by an appropriate promoter. The plasmid does not carry any antibiotic resistance marker genes.

### Example 4: Confirmation of Plasmid Maintenance Based on the Different Reporter Plasmids

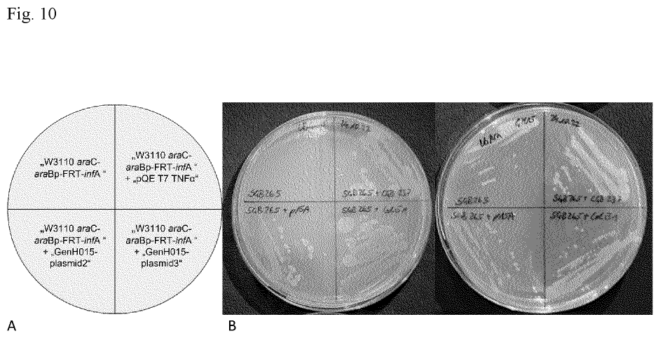

“GenH015-plasmid1”, “GenH015-plasmid2” and “GenH015-plasmid3” Electrocompetent cells were prepared from the platform strain W3110 araC-araBp-FRT-infA as described in Example 3. Approximately 10 ng of the plasmid “pQE-T7 TNFα infA”, “GenH015-plasmid2” and “GenH015-plasmid3” were electroporated into the cells of the platform strains. The cells were resuspended after electroporation in 1 ml LB medium (without antibiotics or L-arabinose) and incubated for two hours at 37° C. with shaking at 800 rpm. After this recovery phase the cells were streaked out on LB agar plates (without antibiotics or L-arabinose) and incubated overnight at 37° C. More than one hundred colonies were visible on the plates the next day. All colonies obtained after electroporation with “GenH015-plasmid2” and “GenH015-plasmid3” displayed the expected violet color indicating the presence of the corresponding plasmid. The three different plasmids tested were all able to complement the platform strain and allow normal growth on LB medium without L-arabinose (FIG. 10).

The γ origin of replication of the broad-host-range plasmid R6K (oriR6Kγ) has the advantage of a very small overall size. Replication of these plasmids requires the π protein encoded by the pir gene. This protein is in general expressed in trans from a chromosomal integrated pir gene.

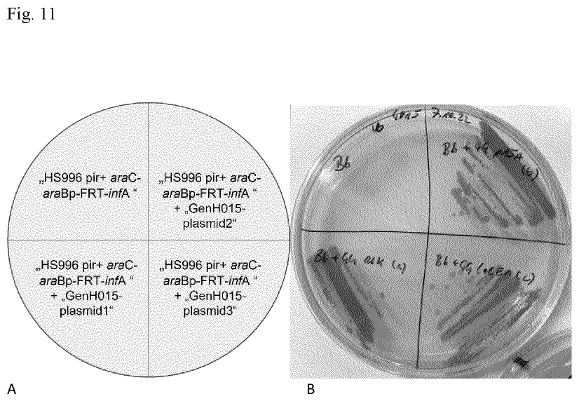

To test whether plasmid maintenance will be obtained with plasmids using an oriR6Kγ the platform strain HS996 pir+ araC-araBp-FRT-infA was transformed with the three reporter plasmids “GenH015-plasmid1”, “GenH015-plasmid2” and “GenH015-plasmid3”. More than one hundred colonies were visible on all plates the next day. All colonies obtained displayed the expected violet color indicating the presence of the corresponding plasmid. The three different plasmids tested were all able to complement the platform strain and allow normal growth on LB medium without L-arabinose (FIG. 11).

To verify a long-term plasmid maintenance in the absence of antibiotics three different combinations of platform strain and plasmid were further analysed: “HS996 pir+ araC-araBp-FRT-infA”/“GenH015-plasmid1”, “T7E2 araC-araBp-FRT-infA”/“GenH015-plasmid2” and “W3110 araC-araBp-FRT-infA”/“GenH015-plasmid3”.

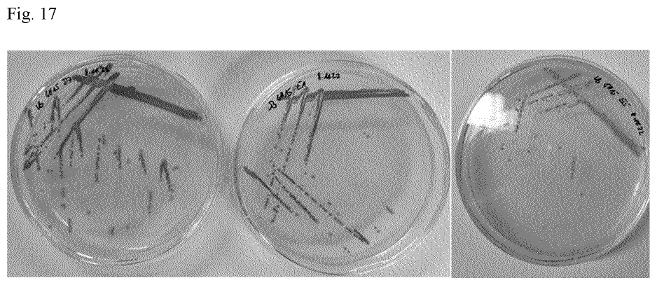

Single colonies of the platform strains carrying the corresponding plasmid were used to inoculate 1.5 ml LB medium. The cultures were incubated overnight at 37° C. with shaking at 800 rpm. The next day, 10 μl of the overnight cultures were taken to inoculate another 1.5 ml LB medium. The cultures were again incubated at 37° C. until the next morning with shaking at 800 rpm. This procedure was repeated for five days. A small aliquot from the last overnight culture was streaked out onto an LB agar plate and incubated at 37° C. overnight to obtain single colonies. All colonies obtained displayed the expected violet color indicating the presence of the corresponding plasmid (FIG. 17). The color of the colonies obtained from the combination “T7E2 araC-araBp-FRT-infA”/“GenH015-plasmid2” is less intense due to the low copy number of the p15A-based plasmid.

This result demonstrates an effective maintenance of the plasmid during long-term passaging without the use of antibiotics.

### Example 5: Preparation of a Platform Strain Based on the Gene frr

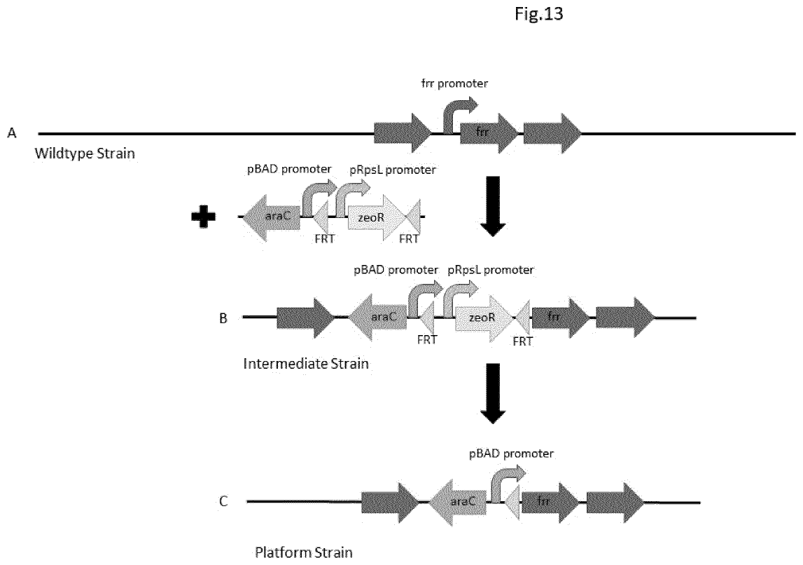

To replace the endogenous frr promoter by an arabinose-inducible promoter, an FRT-flanked zeocin selection marker was linked downstream to the arabinose-inducible promoter sequence, and the corresponding DNA fragment was inserted in the genome via Red/ET recombination at the frr promoter locus. After the replacement of the endogenous frr promoter, an FLP-recombination step was performed, removing the antibiotic resistance marker gene. One FRT site remained either upstream of the inducible promoter in the genome or downstream between the promoter and the start codon of frr (FIG. 13).

a) Replacement of the Endogenous Frr Promoter by an araC-araBp-FRT-zeoR-FRT Cassette

A DNA fragment of 2423 bp in size consisting of araC-araBp linked to an FRT-flanked zeoR resistance marker gene and flanked by homology arms to the frr genomic locus (SEQ ID NO: 29) was amplified using the primers FRT-frr-primer up (SEQ ID NO: 30) and FRT-frr-primer down (SEQ ID NO: 31).

Red/ET recombination (Zhang, Y. et al. 2000; Murphy, K. C., Campellone, K. G. and Poteete, A. R. 2000) was used to insert the obtained PCR product upstream of the frr start codon of E. coli strain HS996, replacing the endogenous promoter. The recombineering reaction was performed as described in the manual of the “Quick & Easy E. coli Gene Deletion Kit” (Gene Bridges, Cat. No. K006) with the exception that 0.4% L-arabinose was added to the medium directly after transformation of the cells with the DNA fragment (SEQ ID NO: 29) to induce expression of frr. Cells were grown for two hours at 37° C. in 1 mL LB medium supplemented with 0.4% L-arabinose. Selection for cells with chromosomal integration of the cassette was performed on LB agar plates containing 0.4% L-arabinose and 25 μg/ml zeocin. The correctly performed knock-in was confirmed by PCR across the modified frr locus and subsequent sequencing of the amplicon.

The zeocin resistance marker was subsequently removed by FLP-recombination as described in Example 1 section bc. The sequence of the genomic locus after the insertion of the arabinose-inducible promoter and the FLP-recombination is given in SEQ ID NO: 32.

### b) Removal of the Resistance Marker Gene by FLP-Recombination

The FLP recombination step was performed as described in the manual of the “Quick & Easy E. coli Gene Deletion Kit” (Gene Bridges, Cat. No. K006) with the exception that 0.4% L-arabinose was added to the medium during all steps. The FLP-expression plasmid “708-FLPe cmR” (Gene Bridges, Cat. No. A105) was chosen for the removal of the resistance marker.

Cells were transformed with the FLP-expression plasmid and grown for two hours at 30° C. in 1 mL LB medium supplemented with 0.4% L-arabinose but without the addition of antibiotics. Selection for cells harboring the FLP-expression plasmid was performed on LB agar plates containing 15 μg/ml chloramphenicol and 0.4% L-arabinose. The plates were incubated overnight at 30° C. The next day, 1 mL LB medium supplemented with 0.4% L-arabinose was inoculated with a single colony and incubated for 3 hours at 30° C. Subsequently the incubation temperature was increased to 37° C. and the incubation was continued for another 3 hours. An aliquot of the culture was streaked out on LB agar plates conditioned with 0.4% L-arabinose and the plates were incubated overnight at 37° C. The obtained colonies were analyzed for FLP-mediated marker removal by assessing for sensitivity to the corresponding antibiotic. The correctly performed removal of the antibiotic resistance marker gene was confirmed by PCR and subsequent sequencing of the amplicon. The sequence of the genomic locus after removal of the zeocin resistance marker gene is given in SEQ ID NO: 33.

### c) Growth Control of the Platform Strain

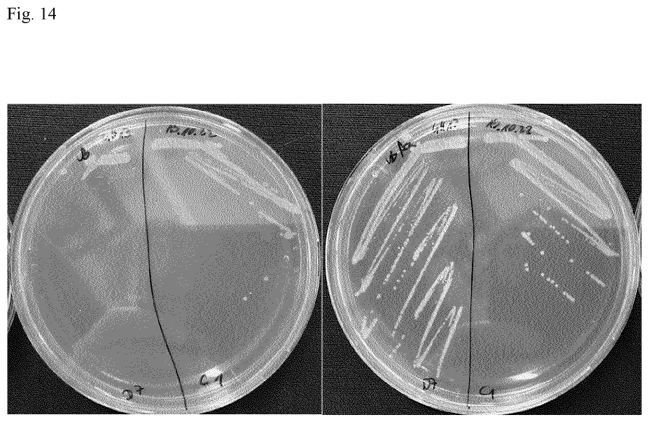

The growth controls of the platform strains were performed as described in Example 1 section ac. Cells were streaked out on LB agar plates with and without the addition of 0.4% L-arabinose to confirm that the obtained platform strain grows only in the presence of L-arabinose (FIG. 14). The platform strain does not carry any antibiotic resistance marker genes in the genome.

d) Preparation of an Expression Plasmid “pQE-T7 TNFα Frr”

Plasmid “pQE-T7 TNFα” (SEQ ID NO: 17) was chosen to confirm that a plasmid encoding the frr gene will be maintained in the platform strains without antibiotic selection. A synthetic DNA fragment consisting of a zeocin resistance marker gene flanked by NotI sites and linked to a genomic frr fragment (SEQ ID NO: 34) was chosen to replace the “cmR-infA” cassette in plasmid “pQE-T7 TNFα cmR-infA” (FIG. 5, SEQ ID NO: 21) using the recombineering proficient E. coli strain GB08-red (Fu et al. 2010).

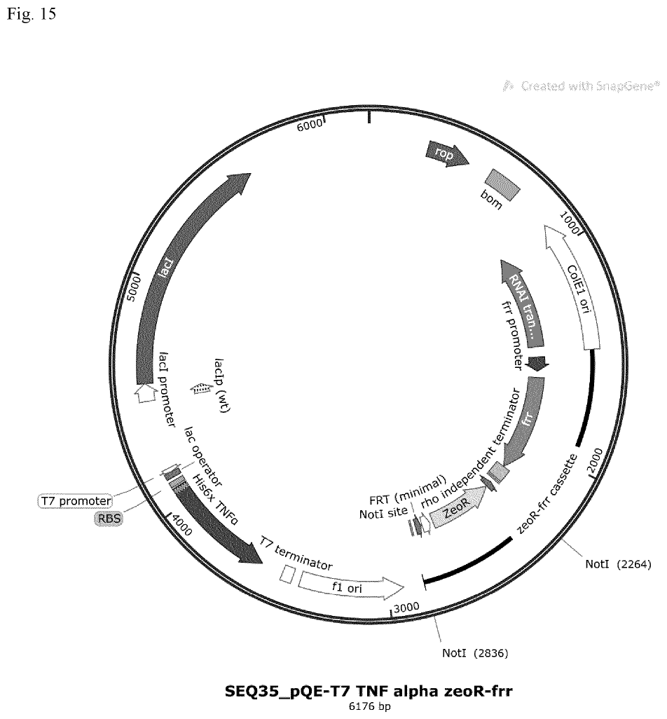

The obtained plasmid “pQE-T7 TNFα zeoR-frr” (FIG. 15, SEQ ID NO: 35) was used as a first test plasmid for the subsequent plasmid maintenance experiments.

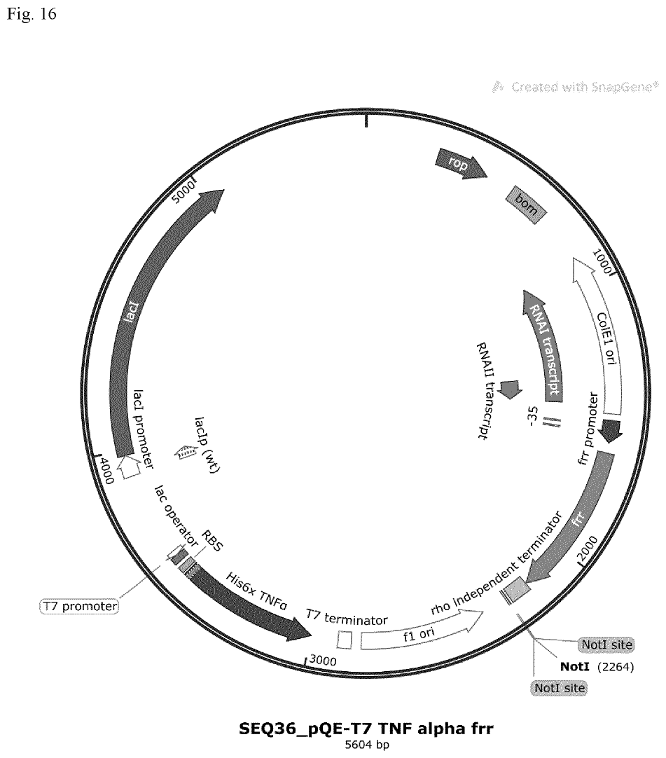

An aliquot of 100 ng plasmid DNA from “pQE-T7 TNFα zeoR-frr” was digested with the restriction enzyme NotI and subsequently religated to obtain a test plasmid without an antibiotic resistance marker gene. This second test plasmid was named “pQE-T7 TNFα frr” (FIG. 16, SEQ ID NO: 36).

### e) Verification of Long-Term Plasmid Maintenance in the Absence of Antibiotics Based on frr

To verify that an frr-based plasmid will be kept in the platform strain as desired, a single colony of the platform strain carrying plasmid “pQE-T7 TNFα zeoR-frr” was used to inoculate 1.5 ml LB medium. The culture was incubated overnight at 37° C. with shaking at 800 rpm. The next day, 10 μl of the overnight culture were taken to inoculate another 1.5 ml LB medium. The culture was again incubated at 37° C. until the next morning with shaking at 800 rpm. This procedure was repeated for five days. A small aliquot from the last overnight culture was streaked out onto an LB agar plate and incubated at 37° C. overnight to obtain single colonies.

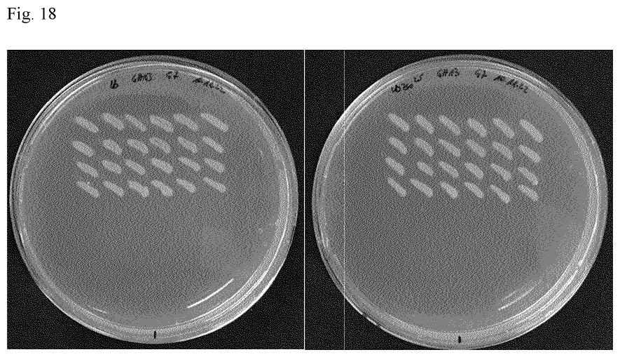

Twenty-four single colonies were streaked out on two LB agar plates, one conditioned with 25 g/ml zeocin and the other one without the addition of antibiotics. The plates were incubated overnight at 37° C. All clones grew on both plates confirming that plasmid “pQE-T7 TNFα zeoR-frr” was maintained in all clones although the cells were grown seven days without the addition of zeocin (FIG. 18). This result demonstrates an effective maintenance of the plasmid during long-term passaging without the use of antibiotics.

### CITED LITERATURE

Szpirer, C. Y., and Milinkovitch, M. C. (2005). “Separate-component-stabilization system for protein and DNA production without the use of antibiotics.” Biotechniques 38, 775-781. https://doi.org/10.2144/05385RR02;
Cranenburgh, R. M., Hanak, J. A., Williams, S. G., and Sherratt, D. J. (2001). “Escherichia coli strains that allow antibiotic-free plasmid selection and maintenance by repressor titration.” Nucleic Acids Research 29, E26. https://doi.org/10.1093/nar/29.5.e26;
Mairhofer, J., Cserjan-Puschmann, M., Striedner, G., Nöbauer, K., Razzazi-Fazeli, E., and Grabherr, R. (2010)., Marker-free plasmids for gene therapeutic applications—Lack of antibiotic resistance gene substantially improves manufacturing process.” Journal Biotechnology. 146, 130-137. https://doi.org/10.1016/j.jbiotec.2010.01.025;
Fiedler, M., and Skerra, A. (2001). “proBA complementation of an auxotrophic E. coli strain improves plasmid stability and expression yield during fermenter production of a recombinant antibody fragment.” Gene 274, 111-118. https://doi.org/10.1016/S0378-1119(01)00629-1;
WO2003/000881
P. Hagg, J. Wa de Pohl, F. Abdulkarim, L. A. Isaksson (2004). “A host/plasmid system that is not dependent on antibiotics and antibiotic resistance genes for stable plasmid maintenance in Escherichia coli.” J. Biotechnol., 111: 17-30
WO2017/097383
WO2018/083116
Brautaset, T., Lale, R., and Valla, S. (2009). “Positively regulated bacterial expression systems”., Microbial Biotechnology 2(1), 15-30. https://doi.org/10.1111/j.1751-7915.2008.00048.x
Shimada, T., Yamazaki, Y., Tanaka, K., and Ishihama, A. (2014), PLoS ONE 9(3): e90447. https://doi.org/10.1371/journal.pone.0090447.
Fu, J., Teucher, M., Anastassiadis, K., Skarnes, W. and Stewart, A. F. (2010). “A recombineering pipeline to make conditional targeting constructs.” Methods in Enzymology 477: 125-144. https://doi.org/10.1016/S0076-6879(10)77008-7;
Chumpolkulwong, N., Hori-Takemoto, C., Hosaka, T., Inaoka, T., Kigawa, T., Shirouzu, M., Ochi, K. and Yokoyama, S. (2004). “Effects of Escherichia coli ribosomal protein S12 mutations on cell-free protein synthesis” European Journal of Biochemistry 271: 1127-1134; https://doi.org/10.1111/j.1432-1033.2004.04016.x;
Zhang, Y., Muyrers, J. P., Testa, G. and Stewart, A. F. (2000). “DNA cloning by homologous recombination in Escherichia coli.” Nature Biotechnology 18: 1314-7; https://doi.org/10.1038/82449;
Murphy, K. C., Campellone, K. G. and Poteete, A. R. (2000). “PCR-mediated gene replacement in Escherichia coli.” Gene 246: 321-30. https://doi.org/10.1016/S0378-1119(00)00071-8;
Kilby, N. J., Snaith, M. R. and Murray, J. A. (1993): “Site-specific recombinases: tools for genome engineering”. Trends in Genetics 9: 413-421; doi: 10.1016/0168-9525(93)90104-p.
Link, A. J., Phillips, D., and Church, G. M. (1997). “Methods for Generating Precise Deletions and Insertion in the Genome of Wild-Type Escherichia coli: Application to Open Reading Frame Characterization.” Journal Biotechnology. 179, 6228-6237. https://doi.org/10.1128/jb.179.20.6228-6237.1997
Alves, C. P. A., Prazeres, D. M. F., and Monteiro, G. A. (2021) “Minicircle Biopharmaceuticals—An Overview of Purification Strategies” Front. Chem. Eng. 2, 612594, https://doi.org/10.3389/fceng.2020.612594
Oliveira, P. H., and Mairhofer, J. (2013). “Marker-free plasmids for biotechnological applications—implications and perspectives” Trends in Biotechnology. 31, 539-547. https://doi.org/10.1016/j.tibtech.2013.06.001
Lu, J., Zhang, F., Xu, S., Fire, A. Z., and Kay, M. A. (2012). “The Extragenic Spacer Length Between the 5a and 3a Ends of the Transgene Expression Cassette Affects Transgene Silencing from Plasmid-based Vectors” Mol. Ther. 20, 2111-2119. https://doi.org/10.1038/mt.2012.65

1. A recombinant bacterial cell comprising in its genome at least one essential gene the endogenous promoter of which has been replaced by a heterologous expression control sequence being inducible by an inducer molecule, such that the expression of said essential gene in the recombinant bacterial cell is dependent on the presence of said inducer molecule.

2. The recombinant bacterial cell of claim 1, wherein said sat least one essential gene (i) is an absolute essential gene which is absolutely required for proper cell growth, proliferation and/or survival even under optimal growth conditions or which is indispensable for DNA replication, RNA synthesis, protein synthesis, central metabolism, cell wall or membrane synthesis or reproduction or (ii) is a combination of essential genes which are in combination absolutely required for proper cell growth, proliferation and/or survival even under optimal growth conditions or which is indispensable for DNA replication, RNA synthesis, protein synthesis, central metabolism, cell wall or membrane synthesis or reproduction.

3. The recombinant bacterial cell of claim 1 or 2, wherein said bacterial cell is from a genus selected from the group consisting of Escherichia, Vibrio, Bacillus, Lactobacillus, Acetobacter, Corynebacterium, Brevibacterium, Pseudomonas, Streptomyces, Gluconobacter, Clostridium, Streptococcus, Zooepidemicus, Basfia, Crytococcus, Rhodotorula, Nocardia, Erwinia, Xanthomonas, Leuconostoc and Klebsiella, preferably, is an E. coli cell, and wherein said at least one essential gene is a gene required for cell growth and/or viability, preferably, selected from the group consisting of: infA, infC/IF-3, dnaJ, dnaK, era, frr, ftsL, ftsN, ftsZ, grpE, mopA, mopB, msbA, nusG, parC, rpsB, secY and trmA.

4. The recombinant bacterial cell of any one of claims 1 to 3, wherein said bacterial cell comprises an expression plasmid which comprises at least one plasmid copy of the at least one essential gene, wherein said plasmid copy is under the control of the expression control sequence which is biologically active in said bacterial cell.

5. The recombinant bacterial cell of claim 4, wherein said expression plasmid comprises at least one further nucleic acid of interest which is required for the recombinant manufacture of a compound of interest in said recombinant bacterial cell.

6. A method for generating the recombinant bacterial cell of any one of claims 1 to 5 comprising the steps of:
(a) exchanging in the genome of a bacterial cell the endogenous promoter of at least one essential gene by a first polynucleotide comprising a selection cassette comprising a promoter that can be active in the bacterial cell operatively linked to
(i) a first selection gene that confers sensitivity for a first selection agent such that the bacterial cell will not grow and/or will die in the presence of said first selection agent, and
(ii) a second selection gene that confers resistance for a second selecting agent such that the bacterial cell will be able to grow in the presence of said second selecting agent,
such that the expression of the at least one essential gene, the first and the second selection gene will become dependent on the said promoter;
(b) selecting a bacterial cell which has integrated into its genome said first polynucleotide using the second selecting agent;
(c) exchanging said first polynucleotide which has been integrated into the genome by a second polynucleotide comprising a heterologous expression control sequence being inducible by an inducer molecule such that the expression of the at least one essential gene becomes dependent on said heterologous expression sequence; and
(d) selecting a bacterial cell which has integrated into its genome said second polynucleotide using the first selection molecule and the inducer molecule.

7. The method of claim 6, wherein said first selection gene is selected from the group consisting of rpsL, sacB, pheS, tetR, galK, thyA, tolC and ccdB and wherein said first selection agent is selected from the group consisting of streptomycin, sucrose, chloro-phenylalanine, chlortetracycline and fusaric acid, galactose, trimethoprim and derivatives thereof, and bacteriocins, preferably, colicins, preferably, colicin A, B, E1, Ia, Ib, N, D, M or E3, more preferably colicin E1.

8. The method of claims 6 or 7, wherein said second selection gene is selected from the group consisting of kmR, ampR, zeoR, tetR, cmR, hygR, specR, and BSD and wherein said second selection agent is selected from the group consisting of kanamycin, ampicillin, zeocin, tetracycline, chloramphenicol, hygromycin, spectinomycin, and blasticidin.

9. The method of any one of claims 6 to 8, wherein said selection cassette is further comprising an restriction enzyme cleavage site at the 3′end of the second selection gene and wherein said method further comprises after step (c) the step of treating the bacterial cell with the restriction enzyme such that a genome of a bacterial cell still comprising the first polynucleotide comprising the selection cassette will be cleaved between the second selection gene and the at least one essential gene while the genome of a bacterial cell having exchanged the first polynucleotide comprising a selection cassette which has been integrated into the genome by a second polynucleotide comprising a heterologous expression control sequence being inducible by an inducer molecule such that the expression of the at least one essential gene becomes dependent on said heterologous expression sequence remains unaffected by the restriction enzyme.

10. A method for generating the recombinant bacterial cell of any one of claims 1 to 5 comprising the steps of:
(a) exchanging in the genome of a bacterial cell the endogenous promoter of at least one essential gene by a polynucleotide comprising
(i) a selection gene that confers resistance for a selection agent, said selection gene being flanked by recognition sites for site specific recombination in the bacterial cell, and
(ii) a heterologous expression control sequence being inducible by an inducer molecule,
such that the expression of the at least one essential gene becomes dependent on said heterologous expression control sequence and such that the bacterial cell will grow in the presence of said inducer molecule and in the presence of said selection agent;
(b) selecting a bacterial cell which has integrated into its genome said polynucleotide using the selection agent and the inducer molecule;
(c) removing the selection gene by using a recombinase that is capable of carrying out site specific recombination using the recognition sites flanking the selection gene such that the expression of the at least one essential gene remains dependent on said heterologous expression control sequence; and
(d) selecting a bacterial cell which has integrated into its genome said polynucleotide lacking the selection gene using the inducer molecule.

11. The method of claim 10, wherein said selection gene is selected from the group consisting of ampR, kmR, zeoR, tetR, cmR, hygR, specR, and BSD and wherein said selection agent is selected from the group consisting of: ampicillin, kanamycin, zeocin, tetracycline, chloramphenicol, hygromycin, spectinomycin, and blasticidin.

12. A method for generating the recombinant bacterial cell of any one of claims 1 to 5 comprising the steps of:
(a) exchanging in the genome of a bacterial cell the endogenous promoter of at least one essential gene by a polynucleotide comprising
(i) a heterologous expression control sequence being inducible by an inducer molecule,
(ii) a promoter that can be active in the bacterial cell, and
(iii) a selection gene that confers resistance for a selection agent,
wherein a segment of the polynucleotide comprising (ii) the promoter that can be active in the bacterial cell and (iii) the selection gene but not the heterologous expression control sequence is flanked by recognition sites for site specific recombination in the bacterial cell,
such that the expression of the at least one essential gene will become dependent on the promoter that can be active in the bacterial cell;
(b) selecting a bacterial cell which has integrated into its genome said polynucleotide using the selection agent;
(c) removing the segment of the polynucleotide comprising the promoter that can be active in the bacterial cell and the selection gene by using a recombinase that is capable of carrying out site specific recombination using the recognition sites flanking the segment of the polynucleotide such that the expression of the at least one essential gene becomes dependent on said heterologous expression control sequence; and
(d) selecting a bacterial cell which has integrated into its genome said polynucleotide lacking the segment flanked by the recognition sites using the inducer molecule.

13. The method of claim 12, wherein said selection gene is selected from the group consisting of: kmR, ampR, zeoR, tetR, cmR, hygR, specR, and BSD and wherein said selection agent is selected from the group consisting of: kanamycin, ampicillin, zeocin, tetracycline, chloramphenicol, hygromycin, spectinomycin, and blasticidin.

14. The method of claim 12 or 13, wherein said recognition sites for site specific recombination in the bacterial cell are FRT or loxP sequences and wherein said recombinase is an FLP or Cre recombinase.

15. The method of any one of claims 12 to 14, wherein said promoter that can be active in the bacterial cell is a constitutively active promoter selected from the group consisting of rpsL promoter, infA promoter, infAp2 promoter, em7 promoter, and cat promoter.

16. The recombinant bacterial cell of any one of claims 1 to 5 or the method of any one of claims 6 to 15 wherein said heterologous expression control sequence being inducible by an inducer molecule is selected from the group consisting of: AraC/PBAD promoter, RhaR-RhaS/rhaBAD promoter, XylS/Pm promoter, NitR/PnitA promoter, ChnR/Pb inducer/promoter, a tetracycline-inducible promoter (tetON/OFF), a lacI/IPTG promoter, an ethanol-inducible promoter (AlcA/AlcR), a steroid-inducible promoter (LexA/XVE), and a vanillate-inducible promoter.

17. The recombinant bacterial cell of any one of claims 1 to 5, the method of any one of claims 6 to 15 or the recombinant bacterial cell or method of claim 16, wherein said inducer molecule is selected from the group consisting of: arabinose, rhamnose, xylose, sucrose, tetracycline, anhydrotetracycline and IPTG.

18. A method for recombinant manufacture of a compound of interest comprising the steps of:
(a) introducing into a recombinant bacterial cell of any one of claims 1 to 5 an expression plasmid which comprises at least one plasmid copy of the at least one essential gene, wherein said plasmid copy is under the control of the expression control sequence which is biologically active in said bacterial cell; and
(b) cultivating the recombinant bacterial cell obtained in step a) in the absence of the inducer molecule.
wherein said expression plasmid comprises at least one further nucleic acid of interest which is required for the recombinant manufacture of a compound of interest in said recombinant bacterial cell.


SEQUENCE LISTING (37 sequences)

SEQ ID NO: 1 - DNA, 1026 bp, synthetic construct
  misc_feature 1..1026: note = rpsL-zeoR cassette
  source 1..1026: mol_type = other DNA
gcggcttagccgtgtgttttcggagtaatgtgccgaacctgtttgttgcgtagggataacagggtaatggcctggtgatgatggcgggatcgttgtatatttcttgacaccttttcggcatcgccctaaaattcggcgtcctcatattgtgtgaggacgttttattacgtgtttacgaagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaaggaggacaatcatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgactcgagcccgttcaaattacggtagtgataccccagaggattagatggccaaagaagac

SEQ ID NO: 2 - DNA, 86 bp, synthetic construct
  misc_feature 1..86: note = CS-primer up
  source 1..86: mol_type = other DNA
gcggcttagccgtgtgttttcggagtaatgtgccgaacctgtttgttgcgtagggataacagggtaatggcctggtgatgatggcg

SEQ ID NO: 3 - DNA, 77 bp, synthetic construct
  misc_feature 1..77: note = CS-primer down
  source 1..77: mol_type = other DNA
gtcttctttggccatctaatcctctggggtatcactaccgtaatttgaacgggctcgagtcagtcctgctcctcggc

SEQ ID NO: 4 - DNA, 1442 bp, synthetic construct
  misc_feature 1..1442: note = araC-araBp-infA knock-in cassette
  source 1..1442: mol_type = other DNA
ctcattaccccgctgccgataaggaatttttcgcgtcaggtaacgcccatcacgaggccctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggagaggaataagtagcagatggccaaagaagacaatattgaaatgcaaggtaccgttcttgaaacgttgcctaataccatgttccgcgtagagttagaaaacggtcacgtggttactg

SEQ ID NO: 5 - DNA, 70 bp, synthetic construct
  misc_feature 1..70: note = CS2-primer up
  source 1..70: mol_type = other DNA
ctcattaccccgctgccgataaggaatttttcgcgtcaggtaacgcccatcacgaggccctgcaaaccct

SEQ ID NO: 6 - DNA, 17 bp, synthetic construct
  misc_feature 1..17: note = CS2-primer down
  source 1..17: mol_type = other DNA
cagtaaccacgtgaccg

SEQ ID NO: 7 - DNA, 2064 bp, synthetic construct
  misc_feature 1..2064: note = genomic infA locus after modification (aat-infA segment)
  source 1..2064: mol_type = other DNA
atcacgcgtggcgtggtcgaagcctaccatcgccttcacgaactcggtcatgcccactccattgaagtctggcgtgaagatgagcttgtcggcggtatgtacggcgtggcccagggaacactattttgtggcgagtccatgttcagccggatggaaaatgcgtctaaaacggcgcttctggtattctgtgaggaatttatcggtcatggcggtaagcttatcgactgccaggtccttaacgatcacacagcatcgcttggtgcctgcgaaattccccgccgggattaccttaattatctcaatcaaatgcgcctcggacgattgccgaataatttctgggtaccacggtgcttgttttcaccacaagaatgaatgttttcggcacatttctccccagagtgttataattgcggtcgcagagttggttacgctcattaccccgctgccgataaggaatttttcgcgtcaggtaacgcccatcacgaggccctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggagaggaataagtagcagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtga

SEQ ID NO: 8 - DNA, 2682 bp, synthetic construct
  misc_feature 1..2682: note = FRT-ampR-FRT-araC-araBp knock-in cassette
  source 1..2682: mol_type = other DNA
ctcattaccccgctgccgataaggaatttttcgcgtcaggtaacgcccatgttcattagggctcgaagttcctatactttctagagaataggaacttctcattaatcctgttctcttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcattgtcaacacgtgctgaagttcctatactttctagagaataggaacttccgccctttagtgagggttaattgtctaagaaaccattattatcatgacattaacctataaaaataggcgtatcacgaggccctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggagaggaataagtagcagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgacc

SEQ ID NO: 9 - DNA, 70 bp, synthetic construct
  misc_feature 1..70: note = FRT-primer up
  source 1..70: mol_type = other DNA
ctcattaccccgctgccgataaggaatttttcgcgtcaggtaacgcccatgttcattagggctcgaagtt

SEQ ID NO: 10 - DNA, 22 bp, synthetic construct
  misc_feature 1..22: note = FRT-primer down
  source 1..22: mol_type = other DNA
ggtcagttcaacagtcactttg

SEQ ID NO: 11 - DNA, 2184 bp, synthetic construct
  misc_feature 1..2184: note = genomic infA locus after modification (aat-infA segment)
  source 1..2184: mol_type = other DNA
atcacgcgtggcgtggtcgaagcctaccatcgccttcacgaactcggtcatgcccactccattgaagtctggcgtgaagatgagcttgtcggcggtatgtacggcgtggcccagggaacactattttgtggcgagtccatgttcagccggatggaaaatgcgtctaaaacggcgcttctggtattctgtgaggaatttatcggtcatggcggtaagcttatcgactgccaggtccttaacgatcacacagcatcgcttggtgcctgcgaaattccccgccgggattaccttaattatctcaatcaaatgcgcctcggacgattgccgaataatttctgggtaccacggtgcttgttttcaccacaagaatgaatgttttcggcacatttctccccagagtgttataattgcggtcgcagagttggttacgctcattaccccgctgccgataaggaatttttcgcgtcaggtaacgcccatgttcattagggctcgaagttcctatactttctagagaataggaacttccgccctttagtgagggttaattgtctaagaaaccattattatcatgacattaacctataaaaataggcgtatcacgaggccctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcaggagaggaataagtagcagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtga

SEQ ID NO: 12 - DNA, 2647 bp, synthetic construct
  misc_feature 1..2647: note = araC-araBp-FRT-kmR-FRT knock-in cassette
  source 1..2647: mol_type = other DNA
cgaataatttctgggtaccacggtgcttgttttcaccacaagaatgaatgttttcggcacatttctccccagagtgttataattgcggtcgcagagttggttacgctcattaccccgctgccgataaggaatttttcgcgtcaggtaacgcccatcacgaggccctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcagaagttcctattctctagaaagtataggaacttcctggtgatgatggcgggatcgttgtatatttcttgacaccttttcggcatcgccctaaaattcggcgtcctcatattgtgtgaggacgttttattacgtgtttacgaagcaaaagctaaaaccaggagctatttaatgggatcggccattgaacaagatggattgcacgcaggttctccggccgcttgggtggagaggctattcggctatgactgggcacaacagacgatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgagaagttcctattctctagaaagtataggaacttcgttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgacc

SEQ ID NO: 13 - DNA, 22 bp, synthetic construct
  misc_feature 1..22: note = FRT2-primer up
  source 1..22: mol_type = other DNA
cgaataatttctgggtaccacg

SEQ ID NO: 14 - DNA, 2114 bp, synthetic construct
  misc_feature 1..2114: note = genomic infA locus after modification (aat-infA segment)
  source 1..2114: mol_type = other DNA
atcacgcgtggcgtggtcgaagcctaccatcgccttcacgaactcggtcatgcccactccattgaagtctggcgtgaagatgagcttgtcggcggtatgtacggcgtggcccagggaacactattttgtggcgagtccatgttcagccggatggaaaatgcgtctaaaacggcgcttctggtattctgtgaggaatttatcggtcatggcggtaagcttatcgactgccaggtccttaacgatcacacagcatcgcttggtgcctgcgaaattccccgccgggattaccttaattatctcaatcaaatgcgcctcggacgattgccgaataatttctgggtaccacggtgcttgttttcaccacaagaatgaatgttttcggcacatttctccccagagtgttataattgcggtcgcagagttggttacgctcattaccccgctgccgataaggaatttttcgcgtcaggtaacgcccatcacgaggccctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcagaagttcctattctctagaaagtataggaacttcgttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtga

SEQ ID NO: 15 - DNA, 219 bp, Escherichia coli
  misc_feature 1..219: note = infA gene
  source 1..219: mol_type = unassigned DNA
atggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctga

SEQ ID NO: 16 - DNA, 1332 bp, Escherichia coli
  misc_feature 1..1332: note = secY gene
  source 1..1332: mol_type = unassigned DNA
atggctaaacaaccgggattagattttcaaagtgccaaaggtggcttaggcgagctgaaacgcagactgctgtttgttatcggtgcgctgattgtgttccgtattggctcttttattccgatccctggtattgatgccgctgtacttgccaaactgcttgagcaacagcgaggcaccatcattgagatgtttaacatgttctctggtggtgctctcagccgtgcttctatctttgctctggggatcatgccgtatatttcggcgtcgatcattatccagctgctgacggtggttcacccaacgttggcagaaattaagaaagaaggggagtctggtcgtcgtaagatcagccagtacacccgctacggtactctggtgctggcaatattccagtcgatcggtattgctaccggtctgccgaatatgcctggtatgcaaggcctggtgattaacccgggctttgcattctacttcaccgctgttgtaagtctggtcacaggaaccatgttcctgatgtggttgggcgaacagattactgaacgaggtatcggcaacggtatttcaatcattatcttcgccggtattgtcgcgggactcccgccagccattgcccatactatcgagcaagcgcgtcaaggcgacctgcacttcctcgtgttgctgttggttgcagtattagtatttgcagtgacgttctttgttgtatttgttgagcgtggtcaacgccgcattgtggtaaactacgcgaaacgtcagcaaggtcgtcgtgtctatgctgcacagagcacacatttaccgctgaaagtgaatatggcgggggtaatcccggcaatcttcgcttccagtattattctgttcccggcgaccatcgcgtcatggttcgggggcggtactggttggaactggctgacaacaatttcgctgtatttgcagcctgggcaaccgctttatgtgttactctatgcgtctgcaatcatcttcttctgtttcttctacacggcgttggttttcaacccgcgtgaaacagcagataacctgaagaagtccggtgcatttgtaccaggaattcgtccgggagagcaaacggcgaagtatatcgataaagtaatgacccgcctgaccctggttggtgcgctgtatattacctttatctgcctgatcccggagttcatgcgtgatgcaatgaaagtaccgttctacttcggtgggacctcactgcttatcgttgttgtcgtgattatggactttatggctcaagtgcaaactctgatgatgtccagtcagtatgagtctgcattgaagaaggcgaacctgaaaggctacggccgataa

SEQ ID NO: 17 - DNA, 5746 bp, synthetic construct
  misc_feature 1..5746: note = pQE-T7 TNF alpha
  source 1..5746: mol_type = other DNA
ccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcgr

SEQ ID NO: 18 - DNA, 68 bp, synthetic construct
  misc_feature 1..68: note = infA-primer up
  source 1..68: mol_type = other DNA
gtttttttgtttgcaagcagcagattacgcgcagaaaaaaaggatctcaacggagtaatgtgccgaac

SEQ ID NO: 19 - DNA, 77 bp, synthetic construct
  misc_feature 1..77: note = infA-primer down
  source 1..77: mol_type = other DNA
gtttatttttctaaatacattcaaatatgtatccgctcatgaattaattcgcggccgcttacgccccgccctgccac

SEQ ID NO: 20 - DNA, 1327 bp, synthetic construct
  misc_feature 1..1327: note = infA-cmR expression cassette
  source 1..1327: mol_type = other DNA
gtttttttgtttgcaagcagcagattacgcgcagaaaaaaaggatctcaacggagtaatgtgccgaacctgtttgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgctgcagcagcacgtgttgacaattaatcatcggcatagtatatcggcatagtataatacgacaaggtgaggaactaaaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagcggccgcgaattaattcatgagcggatacatatttgaatgtatttagaaaaataaac

SEQ ID NO: 21 - DNA, 6036 bp, synthetic construct
  misc_feature 1..6036: note = pQE-T7 TNF cmR-infA
  source 1..6036: mol_type = other DNA
ctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaacggagtaatgtgccgaacctgtttgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgctgcagcagcacgtgttgacaattaatcatcggcatagtatatcggcatagtataatacgacaaggtgaggaactaaaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagcggccgcgaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgcc

SEQ ID NO: 22 - DNA, 5289 bp, synthetic construct
  misc_feature 1..5289: note = pQE-T7 TNF infA
  source 1..5289: mol_type = other DNA
ctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaacggagtaatgtgccgaacctgtttgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgcgaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgcc

SEQ ID NO: 23 - DNA, 3184 bp, synthetic construct
  source 1..3184: mol_type = other DNA
  misc_feature 1..3184: note = plasmid1-cmR
gctgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgctgcagcagcacgtgttgacaattaatcatcggcatagtatatcggcatagtataatacgacaaggtgaggaactaaaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagcggccgcgcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgaggatccttgacggctagctcagtcctaggtacagtgctagctactagagtactagagtactagagattaaagaggagaaatactagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagactcacggtatggacgaattgtacaagcacgacgaattgtctggaggtactgctactgcttctggtatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtagtaataaaaaaaaaaaccccgcttcggcggggttctttttttcagtgcaatttatctcttcaaatgtagcacctgaagtcagccccatctcgag

SEQ ID NO: 24 - DNA, 3417 bp, synthetic construct
  source 1..3417: mol_type = other DNA
  misc_feature 1..3417: note = plasmid2-cmR
gctgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgctgcagcagcacgtgttgacaattaatcatcggcatagtatatcggcatagtataatacgacaaggtgaggaactaaaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagcggccgcgcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgatccttgacggctagctcagtcctaggtacagtgctagctactagagtactagagtactagagattaaagaggagaaatactagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagactcacggtatggacgaattgtacaagcacgacgaattgtctggaggtactgctactgcttctggtatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtagtaataaaaaaaaaaaccccgcttcggcggggttctttttttcagtgcaatttatctcttcaaatgtagcacctgaagtcagccccatctcgag

SEQ ID NO: 25 - DNA, 3596 bp, synthetic construct
  source 1..3596: mol_type = other DNA
  misc_feature 1..3596: note = plasmid3-cmR
gctgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgctgcagcagcacgtgttgacaattaatcatcggcatagtatatcggcatagtataatacgacaaggtgaggaactaaaccatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaagcggccgcgcaacttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagatccttgacggctagctcagtcctaggtacagtgctagctactagagtactagagtactagagattaaagaggagaaatactagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagactcacggtatggacgaattgtacaagcacgacgaattgtctggaggtactgctactgcttctggtatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtagtaataaaaaaaaaaaccccgcttcggcggggttctttttttcagtgcaatttatctcttcaaatgtagcacctgaagtcagccccatctcgag

SEQ ID NO: 26 - DNA, 2437 bp, synthetic construct
  source 1..2437: mol_type = other DNA
  misc_feature 1..2437: note = plasmid1
gctgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgcgcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgaggatccttgacggctagctcagtcctaggtacagtgctagctactagagtactagagtactagagattaaagaggagaaatactagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagactcacggtatggacgaattgtacaagcacgacgaattgtctggaggtactgctactgcttctggtatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtagtaataaaaaaaaaaaccccgcttcggcggggttctttttttcagtgcaatttatctcttcaaatgtagcacctgaagtcagccccatctcgag

SEQ ID NO: 27 - DNA, 2670 bp, synthetic construct
  source 1..2670: mol_type = other DNA
  misc_feature 1..2670: note = plasmid2
gctgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgcgcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgatccttgacggctagctcagtcctaggtacagtgctagctactagagtactagagtactagagattaaagaggagaaatactagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagactcacggtatggacgaattgtacaagcacgacgaattgtctggaggtactgctactgcttctggtatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtagtaataaaaaaaaaaaccccgcttcggcggggttctttttttcagtgcaatttatctcttcaaatgtagcacctgaagtcagccccatctcgag

SEQ ID NO: 28 - DNA, 2849 bp, synthetic construct
  source 1..2849: mol_type = other DNA
  misc_feature 1..2849: note = plasmid3
gctgttgcgatttagcgcgcaaatctttacttatttacagaacttcggcattatcttgccggttcaaattacggtagtgataccccagaggattagatggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctgattgttttaccgcctgatgggcgaagagaaagaacgagtaaaaggtcggtttaaccggcctttttattttgtgatatgtatgaagtactttggaagtataagtccgtaacttgtctcgatgttaattaagtttaaacgcggccgcgcaacttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagatccttgacggctagctcagtcctaggtacagtgctagctactagagtactagagtactagagattaaagaggagaaatactagatggatagcactgagaacgtcatcaagcccttcatgcgcttcaaggtgcacatggagggctccgtgaacggccacgagttcgagatcgagggcgtgggcgagggcaagccctacgagggcacccagaccgccaagctgcaagtgaccaagggcggccccctgcccttcgcctgggacatcctgtccccccagttcttctacggctccaaggcgtacatcaagcaccccgccgacatccccgactacctcaagcagtccttccccgagggcttcaagtgggagcgcgtgatgaacttcgaggacggcggcgtggtgaccgtgacccaggactcctccctgcaggacggcaccctcatctaccacgtgaagttcatcggcgtgaacttcccctccgacggccccgtaatgcagaagaagactctgggctgggagccctccactgagcgcaactacccccgcgacggcgtgctgaagggcgagaaccacatggcgctgaagctgaagggcggcggccactacctgtgtgagttcaagtccatctacatggccaagaagcccgtgaagctgcccggctaccactacgtggactacaagctcgacatcacctcccacaacgaggactacaccgtggtggagcagtacgagcgcgccgaggcccgccaccacctgttccagactcacggtatggacgaattgtacaagcacgacgaattgtctggaggtactgctactgcttctggtatggtgagcaagggcgaggagaataacatggccatcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctaacccccaacttcacctacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgctgaggtcaagaccacctacaaggccaagaagcccgtgcagctgcccggcgcctacatcgtcggcatcaagttggacatcacctcccacaacgaggactacaccatcgtggaactgtacgaacgcgccgagggccgccactccaccggcggcatggacgagctgtacaagtagtaataaaaaaaaaaaccccgcttcggcggggttctttttttcagtgcaatttatctcttcaaatgtagcacctgaagtcagccccatctcgag

SEQ ID NO: 29 - DNA, 2423 bp, synthetic construct
  source 1..2423: mol_type = other DNA
  misc_feature 1..2423: note = araC-araBp-FRT-zeoR-FRT knock-in cassette
tttaccaccgactcagcagcttgcctgcgtggtatcgaaattgaagccgatgtggtgctgaaagcaaccaaagttgacggcgtgtttaccgctgatccggcgaaagatccaaccgcaaccatgtacgagcaactgacttacagcgaagtgctggaaaaagagctgaaagtcatggacctggcggccttcacgctggctcgtgaccataaattaccgattcgtgttttcaatatgaacaaaccgggtgcgctgcgccgtgtggtaatgggtgaaaaagaagggactttaatcacggaataattcccgtgatggataaataagggtaagattccgcgtaagtatcgcgggggcacgaggccctgcaagccctatgctactccgtcaagccgtcaattgtctgattcgttaccaattatgacaacttgacggctacatcattcactttttcttcacaaccagcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgcgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcaggtttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgaggatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcagaagttcctattctctagaaagtataggaacttcctggtgatgatggcgggatcgttgtatatttcttgacaccttttcggcatcgccctaaaattcggcgtcctcatattgtgtgaggacgttttattacgtgtttacgaagcaaaagctaaaaccaggagctatttaatggctaaactgacttctgcagttccggttctgactgctcgtgatgttgctggtgctgttgaattttggactgatcgtctgggtttttctcgtgattttgttgaagatgattttgctggtgttgttcgtgatgatgttactctgtttatttccgctgttcaggatcaggttgttccggataacactctggcttgggtttgggttcgtggtctggatgaactgtatgctgaatggtctgaagttgtttctactaactttcgtgatgcttccggtccggcaatgactgaaattggtgaacagccgtggggtcgtgaatttgcactgcgtgatccggcaggtaactgtgtacattttgtagcagaagaacaggattaggaagttcctattctctagaaagtataggaacttccattaaagaggagaaattaaccgtgattagcgatatcagaaaagatgctgaagtacgcatggacaaatgcgtagaagcgttcaaaacccaaatcagcaaaatacgcacgggtcgtgcttctcccagcctgctggatggcattgtcgtggaatattacggcacgccgacgccgctgcgtcagctggcaagcgtaacggtagaagattcccgtacactgaaaat

SEQ ID NO: 30 - DNA, 19 bp, unidentified
  source 1..19: mol_type = other DNA
  misc_feature 1..19: note = FRT-frr-primer up
tttaccaccgactcagcag

SEQ ID NO: 31 - DNA, 23 bp, unidentified
  source 1..23: mol_type = other DNA
  misc_feature 1..23: note = FRT-frr-primer down
attttcagtgtacgggaatcttc

SEQ ID NO: 32 - DNA, 3432 bp, Escherichia coli
  source 1..3432: mol_type = unassigned DNA
  misc_feature 1..3432: note = pyrH-araC-araBp-FRT-zeo-FRT.frr
ttatcaaaaaggagccgcctgagggcggcttctttttgtgcccatcttgtaaattcagctaacccttgtggggctgcgctgaaaagcgacgtacaatgtcgctagtattaattcatttcaatcgttgacagtctcaggaaagaaacatggctaccaatgcaaaacccgtctataaacgcattctgcttaagttgagtggcgaagctctgcagggcactgaaggcttcggtattgatgcaagcatactggatcgtatggctcaggaaatcaaagaactggttgaactgggtattcaggttggtgtggtgattggtgggggtaacctgttccgtggcgctggtctggcgaaagcgggtatgaaccgcgttgtgggcgaccacatggggatgctggcgaccgtaatgaacggcctggcaatgcgtgatgcactgcaccgcgcctatgtgaacgctcgtctgatgtccgctattccattgaatggcgtgtgcgacagctacagctgggcagaagctatcagcctgttgcgcaacaaccgtgtggtgatcctctccgccggtacaggtaacccgttctttaccaccgactcagcagcttgcctgcgtggtatcgaaattgaagccgatgtggtgctgaaagcaaccaaagttgacggcgtgtttaccgctgatccggcgaaagatccaaccgcaaccatgtacgagcaactgacttacagcgaagtgctggaaaaagagctgaaagtcatggacctggcggccttcacgctggctcgtgaccataaattaccgattcgtgttttcaatatgaacaaaccgggtgcgctgcgccgtgtggtaatgggtgaaaaagaagggactttaatcacggaataattcccgtgatggataaataagggtaagattccgcgtaagtatcgcgggggcacgaggccctgcaagccctatgctactccgtcaagccgtcaattgtctgattcgttaccaattatgacaacttgacggctacatcattcactttttcttcacaaccagcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgcgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcaggtttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgaggatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcagaagttcctattctctagaaagtataggaacttcctggtgatgatggcgggatcgttgtatatttcttgacaccttttcggcatcgccctaaaattcggcgtcctcatattgtgtgaggacgttttattacgtgtttacgaagcaaaagctaaaaccaggagctatttaatggctaaactgacttctgcagttccggttctgactgctcgtgatgttgctggtgctgttgaattttggactgatcgtctgggtttttctcgtgattttgttgaagatgattttgctggtgttgttcgtgatgatgttactctgtttatttccgctgttcaggatcaggttgttccggataacactctggcttgggtttgggttcgtggtctggatgaactgtatgctgaatggtctgaagttgtttctactaactttcgtgatgcttccggtccggcaatgactgaaattggtgaacagccgtggggtcgtgaatttgcactgcgtgatccggcaggtaactgtgtacattttgtagcagaagaacaggattaggaagttcctattctctagaaagtataggaacttccattaaagaggagaaattaaccgtgattagcgatatcagaaaagatgctgaagtacgcatggacaaatgcgtagaagcgttcaaaacccaaatcagcaaaatacgcacgggtcgtgcttctcccagcctgctggatggcattgtcgtggaatattacggcacgccgacgccgctgcgtcagctggcaagcgtaacggtagaagattcccgtacactgaaaatcaacgtgtttgatcgttcaatgtctccggccgttgaaaaagcgattatggcgtccgatcttggcctgaacccgaactctgcgggtagcgacatccgtgttccgctgccgccgctgacggaagaacgtcgtaaagatctgaccaaaatcgttcgtggtgaagcagaacaagcgcgtgttgcagtacgtaacgtgcgtcgtgacgcgaacgacaaagtgaaagcactgttgaaagataaagagatcagcgaagacgacgatcgccgttctcaggacgatgtacagaaactgactgatgctgcaatcaagaaaattgaagcggcgctggcagacaaagaagcagaactgatgcagttctgatttcttgaacgacaaaaacgccgctcagtagatccttgcggatcggctggcggcgttttgctttttattctgtctcaac

SEQ ID NO: 33 - DNA, 2888 bp, unidentified
  source 1..2888: mol_type = unassigned DNA
  misc_feature 1..2888: note = pyrH-araC-araBp-FRT-frr
ttatcaaaaaggagccgcctgagggcggcttctttttgtgcccatcttgtaaattcagctaacccttgtggggctgcgctgaaaagcgacgtacaatgtcgctagtattaattcatttcaatcgttgacagtctcaggaaagaaacatggctaccaatgcaaaacccgtctataaacgcattctgcttaagttgagtggcgaagctctgcagggcactgaaggcttcggtattgatgcaagcatactggatcgtatggctcaggaaatcaaagaactggttgaactgggtattcaggttggtgtggtgattggtgggggtaacctgttccgtggcgctggtctggcgaaagcgggtatgaaccgcgttgtgggcgaccacatggggatgctggcgaccgtaatgaacggcctggcaatgcgtgatgcactgcaccgcgcctatgtgaacgctcgtctgatgtccgctattccattgaatggcgtgtgcgacagctacagctgggcagaagctatcagcctgttgcgcaacaaccgtgtggtgatcctctccgccggtacaggtaacccgttctttaccaccgactcagcagcttgcctgcgtggtatcgaaattgaagccgatgtggtgctgaaagcaaccaaagttgacggcgtgtttaccgctgatccggcgaaagatccaaccgcaaccatgtacgagcaactgacttacagcgaagtgctggaaaaagagctgaaagtcatggacctggcggccttcacgctggctcgtgaccataaattaccgattcgtgttttcaatatgaacaaaccgggtgcgctgcgccgtgtggtaatgggtgaaaaagaagggactttaatcacggaataattcccgtgatggataaataagggtaagattccgcgtaagtatcgcgggggcacgaggccctgcaagccctatgctactccgtcaagccgtcaattgtctgattcgttaccaattatgacaacttgacggctacatcattcactttttcttcacaaccagcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgcgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcaggtttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgaggatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcagaagttcctattctctagaaagtataggaacttccattaaagaggagaaattaaccgtgattagcgatatcagaaaagatgctgaagtacgcatggacaaatgcgtagaagcgttcaaaacccaaatcagcaaaatacgcacgggtcgtgcttctcccagcctgctggatggcattgtcgtggaatattacggcacgccgacgccgctgcgtcagctggcaagcgtaacggtagaagattcccgtacactgaaaatcaacgtgtttgatcgttcaatgtctccggccgttgaaaaagcgattatggcgtccgatcttggcctgaacccgaactctgcgggtagcgacatccgtgttccgctgccgccgctgacggaagaacgtcgtaaagatctgaccaaaatcgttcgtggtgaagcagaacaagcgcgtgttgcagtacgtaacgtgcgtcgtgacgcgaacgacaaagtgaaagcactgttgaaagataaagagatcagcgaagacgacgatcgccgttctcaggacgatgtacagaaactgactgatgctgcaatcaagaaaattgaagcggcgctggcagacaaagaagcagaactgatgcagttctgatttcttgaacgacaaaaacgccgctcagtagatccttgcggatcggctggcggcgttttgctttttattctgtctcaac

SEQ ID NO: 34 - DNA, 1484 bp, synthetic construct
  source 1..1484: mol_type = other DNA
  misc_feature 1..1484: note = zeoR-frr-expression cassette
gtttttttgtttgcaagcagcagattacgcgcagaaaaaaaggatctcaagcccggggccaaaatggcaaataaaatagcctaataatccagacgattacccgtaatatgtttaatcagggctatacttagcacacttccactgtgtgtgactgtctggtctgactgagacaagttttcaaggattcgtaacgtgattagcgatatcagaaaagatgctgaagtacgcatggacaaatgcgtagaagcgttcaaaacccaaatcagcaaaatacgcacgggtcgtgcttctcccagcctgctggatggcattgtcgtggaatattacggcacgccgacgccgctgcgtcagctggcaagcgtaacggtagaagattcccgtacactgaaaatcaacgtgtttgatcgttcaatgtctccggccgttgaaaaagcgattatggcgtccgatcttggcctgaacccgaactctgcgggtagcgacatccgtgttccgctgccgccgctgacggaagaacgtcgtaaagatctgaccaaaatcgttcgtggtgaagcagaacaagcgcgtgttgcagtacgtaacgtgcgtcgtgacgcgaacgacaaagtgaaagcactgttgaaagataaagagatcagcgaagacgacgatcgccgttctcaggacgatgtacagaaactgactgatgctgcaatcaagaaaattgaagcggcgctggcagacaaagaagcagaactgatgcagttctgatttcttgaacgacaaaaacgccgctcagtagatccttgcggatcggctggcggcgttttgctttttattctgtctcaactctggatggcggccgctagcacggagttcattagggctcgaagttcctatactttctagagaataggaacttctcattaatcctgttcttctgctacaaaatgtacacagttacctgccggatcacgcagtgcaaattcacgaccccacggctgttcaccaatttcagtcattgccggaccggaagcatcacgaaagttagtagaaacaacttcagaccattcagcatacagttcatccagaccacgaacccaaacccaagccagagtgttatccggaacaacctgatcctgaacagcggaaataaacagagtaacatcatcacgaacaacaccagcaaaatcatcttcaacaaaatcacgagaaaaacccagacgatcagtccaaaattcaacagcaccagcaacatcacgagcagtcagaaccggaactgcagaagtcagtttagccatggtttagttcctcaccttgtcgtattatactatgccgatatactatgccgatgattaattgtcaacacgtgctgaagttcctatactttctagagaataggaacttccgccctttagtgagggttaattgcggccgcgaattaattcatgagcggatacatatttgaatgtatttagaaaaataaacaaataggggttccgcgc

SEQ ID NO: 35 - DNA, 6176 bp, synthetic construct
  source 1..6176: mol_type = other DNA
  misc_feature 1..6176: note = pQE-T7 TNF alpha zeoR-frr
ctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagcccggggccaaaatggcaaataaaatagcctaataatccagacgattacccgtaatatgtttaatcagggctatacttagcacacttccactgtgtgtgactgtctggtctgactgagacaagttttcaaggattcgtaacgtgattagcgatatcagaaaagatgctgaagtacgcatggacaaatgcgtagaagcgttcaaaacccaaatcagcaaaatacgcacgggtcgtgcttctcccagcctgctggatggcattgtcgtggaatattacggcacgccgacgccgctgcgtcagctggcaagcgtaacggtagaagattcccgtacactgaaaatcaacgtgtttgatcgttcaatgtctccggccgttgaaaaagcgattatggcgtccgatcttggcctgaacccgaactctgcgggtagcgacatccgtgttccgctgccgccgctgacggaagaacgtcgtaaagatctgaccaaaatcgttcgtggtgaagcagaacaagcgcgtgttgcagtacgtaacgtgcgtcgtgacgcgaacgacaaagtgaaagcactgttgaaagataaagagatcagcgaagacgacgatcgccgttctcaggacgatgtacagaaactgactgatgctgcaatcaagaaaattgaagcggcgctggcagacaaagaagcagaactgatgcagttctgatttcttgaacgacaaaaacgccgctcagtagatccttgcggatcggctggcggcgttttgctttttattctgtctcaactctggatggcggccgctagcacggagttcattagggctcgaagttcctatactttctagagaataggaacttctcattaatcctgttcttctgctacaaaatgtacacagttacctgccggatcacgcagtgcaaattcacgaccccacggctgttcaccaatttcagtcattgccggaccggaagcatcacgaaagttagtagaaacaacttcagaccattcagcatacagttcatccagaccacgaacccaaacccaagccagagtgttatccggaacaacctgatcctgaacagcggaaataaacagagtaacatcatcacgaacaacaccagcaaaatcatcttcaacaaaatcacgagaaaaacccagacgatcagtccaaaattcaacagcaccagcaacatcacgagcagtcagaaccggaactgcagaagtcagtttagccatggtttagttcctcaccttgtcgtattatactatgccgatatactatgccgatgattaattgtcaacacgtgctgaagttcctatactttctagagaataggaacttccgccctttagtgagggttaattgcggccgcgaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgcc

SEQ ID NO: 36 - DNA, 5604 bp, synthetic construct
  source 1..5604: mol_type = other DNA
  misc_feature 1..5604: note = pQE-T7TNF alpha frr
ctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagcccggggccaaaatggcaaataaaatagcctaataatccagacgattacccgtaatatgtttaatcagggctatacttagcacacttccactgtgtgtgactgtctggtctgactgagacaagttttcaaggattcgtaacgtgattagcgatatcagaaaagatgctgaagtacgcatggacaaatgcgtagaagcgttcaaaacccaaatcagcaaaatacgcacgggtcgtgcttctcccagcctgctggatggcattgtcgtggaatattacggcacgccgacgccgctgcgtcagctggcaagcgtaacggtagaagattcccgtacactgaaaatcaacgtgtttgatcgttcaatgtctccggccgttgaaaaagcgattatggcgtccgatcttggcctgaacccgaactctgcgggtagcgacatccgtgttccgctgccgccgctgacggaagaacgtcgtaaagatctgaccaaaatcgttcgtggtgaagcagaacaagcgcgtgttgcagtacgtaacgtgcgtcgtgacgcgaacgacaaagtgaaagcactgttgaaagataaagagatcagcgaagacgacgatcgccgttctcaggacgatgtacagaaactgactgatgctgcaatcaagaaaattgaagcggcgctggcagacaaagaagcagaactgatgcagttctgatttcttgaacgacaaaaacgccgctcagtagatccttgcggatcggctggcggcgttttgctttttattctgtctcaactctggatggcggccgcgaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtcttactacagcgcaataatgccaaaatacacctggccgctttccgcaaaatccagataatccggacggttaatttccgcgctcagacgatcgcctttttccagctgaaacacgccgcccagataaatcggttcataccacggtttcgcttccgcgccttccggggtttcacgctggcacgggcttttaatcgcagacagcaggttcactttggtctgatagctcaccgcaatacggctaatggtatgggtcagcagcacatgggtgctcgggcagccctggcctttaaacagcacctggctataaatcagatacaggccttcgctcggcacaaccagctgattatcacgcagttccacgccgttggccagcagcgcattcgcacgacggttcagccattgcagctggccttccgcctgcggattcgccaccacatgcgccaccggtttatcgctcggggtacggctgctgctacgaacctgtttcatatggtgatggtgatggtgtttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgcc

SEQ ID NO: 37 - DNA, 558 bp, unidentified
  source 1..558: mol_type = unassigned DNA
  misc_feature 1..558: note = genomic frr locus
gtgattagcgatatcagaaaagatgctgaagtacgcatggacaaatgcgtagaagcgttcaaaacccaaatcagcaaaatacgcacgggtcgtgcttctcccagcctgctggatggcattgtcgtggaatattacggcacgccgacgccgctgcgtcagctggcaagcgtaacggtagaagattcccgtacactgaaaatcaacgtgtttgatcgttcaatgtctccggccgttgaaaaagcgattatggcgtccgatcttggcctgaacccgaactctgcgggtagcgacatccgtgttccgctgccgccgctgacggaagaacgtcgtaaagatctgaccaaaatcgttcgtggtgaagcagaacaagcgcgtgttgcagtacgtaacgtgcgtcgtgacgcgaacgacaaagtgaaagcactgttgaaagataaagagatcagcgaagacgacgatcgccgttctcaggacgatgtacagaaactgactgatgctgcaatcaagaaaattgaagcggcgctggcagacaaagaagcagaactgatgcagttctga